(19) United States
(12) Patent Application Publication
(10) Pub. No.: US 2025/0282876 A1
(43) Pub. Date: Sep. 11, 2025

(54) STABLE Fc FRAGMENTS, CONJUGATES, COMPOSITIONS, AND METHODS OF USE

(71) Applicant: PURDUE RESEARCH FOUNDATION, West Lafayette, IN (US)

(72) Inventors: Philip Low, West Lafayette, IN (US); Ananda Kumar Kanduluru, West Lafayette, IN (US); Madduri Srinivasarao, West Lafayette, IN (US); Imrul Shahriar, West Lafayette, IN (US)

(21) Appl. No.: 18/858,135

(22) PCT Filed: Apr. 19, 2023
(86) PCT No.: PCT/US2023/065925

§ 371 Date: Oct. 18, 2024

Related U.S. Application Data
(60) Provisional application No. 63/332,370, filed on Apr. 19, 2022.

(51) Int. Cl. C07K 16/28; A61K 47/10; A61K 47/68; A61P 31/16

(52) U.S. Cl. CPC C07K 16/283; A61K 47/10; A61K 47/6803; A61K 47/6849; A61P 31/16; C07K 2317/524; C07K 2317/526; C07K 2317/732; C07K 2317/92

(57) ABSTRACT

Stable monomeric Fc (mFc) fragments, conjugates and compositions comprising such mFc fragments, and methods of use thereof.

### PRIORITY

This application is related to and claims the priority benefit of U.S. Provisional Patent Application No. 63/332,370 filed Apr. 19, 2022. The content of the aforementioned application is hereby incorporated by reference in its entirety into this disclosure.

### TECHNICAL FIELD

The present disclosure relates to Fc (fragment crystallizable domain) fragments, conjugates comprising them, compositions, and methods of use.

### BACKGROUND

The most well-known Fc-mediated (fragment crystallizable domain-mediated) antibody effector functions are antibody-dependent cell-mediated cytotoxicity (ADCC), antibody-dependent cellular phagocytosis (ADCP), and complement-dependent cytotoxicity (CDC). In addition, antibodies have been found to mediate inflammation and immunomodulation through the induction of cellular differentiation and activation. These mechanisms can either protect viral replication or enhance infected cell clearance through the antibody-mediated effector functions against virally infected cells, such as viral glycoprotein shedding, viral glycoprotein internalization, antibody cooperativity, and antibody glycosylation.

A family of receptors that recognize the Fc domain of immunoglobulin G (IgG) molecules is known as the FcRn and FcγRs family. Both FcRn and FcγRs are important for the recognition and elimination of pathogens by the immune system. Several studies have established a correlation between Fc:FcγRs affinities and selectivity, and the cytotoxic functions of immune effector cells engaged by immune complexes (ICs).

The human gamma receptors (FcγRs) are FcγRI (CD64), FcγRIIa (CD32a), FcγRIIb (CD32b), FcγRIIc (CD32c), FcγRIIIa (CD16a), and FcγRIIIb (CD16b), and these receptors are expressed at different levels on the surfaces of various immune cells. The FcγRIIIa is a key surface receptor in terms of its contribution to ADCC activity. FcγRIIIa is found on the surfaces of natural killer (NK) cells, macrophages, monocytes, mast cells, eosinophils, and dendritic cells. However, it is the only FcγR expressed by NK cells. Humans express two FcγRIIIa allotypes that differ in a single amino acid at position 158; the residue can be either valine (V) or phenylalanine (F), whereby the isoform with V at position 158 has high affinity for the Fc domain of IgG1, and the one with F at position 158 has low affinity. Engagement of the high affinity FcγRIIIa-V158 by ICs can result in a stronger in vitro cytotoxic potency than that of FcγRIIIa-F158. Fc-engineered antibodies with improved affinity to FcγRIIIa can show higher therapeutic efficacy comparative to native Fc because they can prime and activate NK cells more efficiently.

ADCP activity is known to be triggered by FcγRIIa intracellular signaling. Glycoengineered antibodies can exhibit enhanced affinity toward FcγRIIa, resulting in increased ADCP activity. Mainly, Fc variants displaying high affinity for the FcγRIIa-R131 isoform and high selectivity for FcγRIIa over FcγRIIb have been shown to mediate improved ADCP activity. The mechanism of phagocytes can be engaged by either complement receptor. Infected cells can also be eliminated by CDC as well as by ADCC and/or ADCP mediated by FcγR bearing effector cells.

Almost all FDA-approved therapeutic antibodies are full-size IgG1 antibodies about 150 kDa in size. Dimitrov, Engineered CH2 domains (nanoantibodies), mAbs 1 (1): 26-28 (2009). The same is true for most antibodies in clinical trials. The few exceptions to this are fragment antigen-binding regions (Fabs).

Full-size antibodies present fundamental problems for therapy. One problem is they can exhibit poor tissue penetration, such as poor solid tumor penetration. Dimitrov (2009), supra. Another problem is poor or no binding to regions on the surfaces of some molecules, such as the envelope glycoprotein of human immunodeficiency virus (HIV). Dimitrov (2009), supra.

Efforts have focused on developing therapeutic scaffolds that are smaller in size and higher in stability. For example, Dimitrov proposed the use of the immunoglobulin (Ig) C_{H}2 domain (for IgG, IgA and IgD) and the C_{H}3 domain (for IgE and IgM) as scaffolds. Dimitrov (2009), supra; Gong et al., Shortened engineered human antibody C_{H}2 domains: Increased stability and binding to the human neonatal receptor, J Biological Chem 286 (31): 27288-27293 (2011).

C_{H}2 in particular is critical for Ig effector function, has very weak carbohydrate-mediated interchain protein-protein interactions, and, when isolated, can exist as a stable monomer. The half-life of human C_{H}2 in rabbits (˜70 hrs) is much longer than that of C_{H}3 and Fab (˜15 hrs) and might function to trigger the complement system. Yasmeen et al., The structure and function of immunoglobulin domains: IV. The distribution of some effector functions among the Cγ2 and Cγ3 homology regions of human immunoglobulin G, J Immunology 116 (2): 518-526 (1976); Ellerson et al., A fragment corresponding to the C_{H}2 region of immunoglobulin G (IgG) with complement fixing activity, FEBS Letters 24 (3): 318-322 (1972).

Libraries can be constructed using C_{H}2 and engineered C_{H}2 domains with improved stability as scaffolds for diverse binders to various antigens; such binders can also confer effector functions. Dimitrov (2009), supra. The C_{H}2 domain is the smallest independently folded antibody domain that can be engineered to contain simultaneously antigen-binding sites and binding sites mediating effector and stability functions. Such engineered C_{H}2 domains are called nanoantibodies (nAbs) to distinguish them from engineered V_{H} and V_{L} domains and nanobodies (i.e., camelid single variable domain on a heavy chain (VHH)). Dimitrov (2009), supra.

And, while unstable to thermally induced unfolding, the stability of the C_{H}2 domain can be improved at least by the addition of a disulfide bond between the A and G strands. Gong et al., Engineered human antibody constant domains with increased stability, J Biological Chem 284 (21): 14203-14210 (2009). Further removal of seven N-terminal residues, which crystal structure and NMR data suggest exist as a random coil, also results in a shortened, highly soluble C_{H}2 monomer with remarkable stability, a higher melting temperature, and increased resistance to protease digestion. Gong et al. (2011), supra.

This increase in stability was not accompanied by a disruption in conformation; however, removal of the N-terminal residues can negatively impact binding to Fcγ receptors and related effector functions. Gong (2011), supra. Another disadvantage of the shortened C_{H}2 is that it lacks the binding motif from the C_{H}3 domain which is involved in interactions with the neonatal Fc receptor (FcRn); consequently, C_{H}2 has a shorter serum half-life (Ying et al., Soluble monomeric IgG1 Fc, J Biological Chem 287 (23): 19399-19408 (2012). Binding to human FcRn is comparable to that of Fc, and high solubility appears to be maintained when the C_{H}3 domain is retained with the C_{H}2 domain (C_{H}2-C_{H}3 monomer or monomeric Fc (mFc)). Ying et al. (2012), supra. The half-life of the mFc is increased because it can bind to FcRn in the acidic environment of the endosome after internalization and then be recycled to the cell surface and released into circulation. See, e.g., Rath et al., Fc-fusion proteins and FcRn: structural insights for longer-lasting and more effective therapeutics, Critical Reviews in Biotechnology 35 (2): 235-254 (2015).

Soluble monomeric IgG1 C_{H}3 also has been engineered to build a library of targeting ligands. The ligands have higher pH-dependent binding affinity to FcRn than their C_{H}2 counterparts. See, e.g., Ying et al., Interactions of IgG1 C_{H}2 and C_{H}3 domains with FcRN, Frontiers in Immunology 5: article 146 (Apr. 2, 2014). The dimeric forms of C_{H}2 and C_{H}3 did not exhibit pH-dependent binding to FcRn

In contrast to wild-type (i.e., dimeric) Fc, monomeric IgG1 Fc (mFc) binds FcγR1, but not FcγRIIIa, with very high affinity. Ying et al., Monomeric IgG1 Fc molecules displaying unique Fc receptor interactions that are expoitable to treat inflammation-mediated diseases, mAbs 6 (5): 1201-1210 (2014). mFc binds FcRn the same as dimeric Fc; mFc and dimeric Fc also have similar pharmacokinetics in mice. When mFc was fused to the 38 kDa Pseudomonas exotoxin A fragment (PE38), the fusion protein killed FcγR1-positive macrophage-like U937 cells but not FcγR1-negative cells.

Fusion of a short FcRn-binding motif derived from IgG1 C_{H}3 to C_{H}2-derived engineered antibody domains (eAds) can enhance binding to FcRn with strict pH dependency. Ying et al., Engineered antibody domains with significantly increased transcytosis and half-life in macaques mediated by FcRn, mAbs 7:5, 922-930 (2015). The increased affinity can result in significantly enhanced FcRn-mediated epithelial transcytosis and prolonged elimination half-life in cynomolgus macaques. Thus, the half-life of isolated eAds can be prolonged by increasing their binding to FcRn while maintaining their small size. Ying et al. (2015), supra.

Accordingly, the increased binding affinity to FcRn, strict pH dependency of binding, and the dissociation rate (k_{off}) of the IgG-FcRn interaction contribute to long half-lives in vivo. Introduction of three mutations (IKS vs. KAK) at the C-terminus of an autonomous human IgG1 C_{H}2 domain can further increase stability and aggregation resistance. Gao et al., Optimization of the C-terminus of an autonomous human IgG1 C_{H}2 domain for stability and aggregation resistance, Molecular Pharmaceutics 16 (8): 3647-3656 (2019).

What is needed is IgG1 Fc fragments with increased stability and better immune effector function. This and other objects and advantages, as well as inventive features, will be apparent from the detailed description provided herein.

SEQUENCE LISTINGS
The sequences herein (SEQ ID NOS: 1-15) are also provided in computer-readable form
encoded in a file filed herewith and incorporated herein by reference. The information recorded
in computer-readable form is identical to the written Sequence Listings provided herein (e.g.,
pursuant to the United States Code of Federal Regulations 37 C.F.R. § 1.821(f)).
SEQ ID NO: 1 is an amino acid sequence for an N-terminus end of a monomeric Fc(mFc)
fragment (amino acids 216-230 of wild-type): EPKSGDKTHTSPPGP.
SEQ ID NO: 2 is an amino acid sequence for an FcRn binding motif from a C_{H}3 domain
of a monomeric Fc fragment (amino acids 350-354 of wild-type): HNHYT.
SEQ ID NO: 3 is an amino acid sequence for a peptide linker between the C_{H}2 domain
and the FcRn binding motif from a C_{H}3 domain (SEQ ID NO: 2) (amino acids 341-349 of wild-
type): FGNCAYLRPHNHYT.
SEQ ID NO: 4 is an amino acid sequence of a wild-type Fc region of human IgG1, wherein
SEQ ID NO: 1 is underlined at the N-terminus (EV-Fc-WT; aa 216-447):
EPKSGDKTHTSPPGPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGK.
SEQ ID NO: 5 is an amino acid sequence of a mutated version of the wild-type Fc region
of human IgG1 (SEQ ID NO: 4) comprising a GASDALIE mutation, wherein SEQ ID NO: 1 is
underlined at the N-terminus (EV-Fc-GASDALIE; aa 216-447):
EPKSGDKTHTSPPGPAPELLAGPDVFLFPPKPKDTLMISRTPEVTCVVVDVSHEDPEVKF
NWYVDGVEVHNAKTKPREEQYNSTYRVVSVLTVLHQDWLNGKEYKCKVSNKALPLP
EEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSPGK.
SEQ ID NO: 6 is an amino acid sequence of a modified Fc region of human IgG1 (SEQ
ID NO: 4) comprising a DLE mutation, wherein IKAROS family zinc finger 1 (IKS) is substituted
at amino acid positions corresponding to amino acids 338-340 of the wild-type Fc region (EV-Fc-
DLE; aa 216-447):
EPKSGDKTHTSPPGPAPELLGGPDVFLFPPKPKDTLMISRTPEVTCVVVDVSHEDPEVKF
NWYVDGVEVHNAKTKPREEQYNSTYRVVSVLTVLHQDWLNGKEYKCKVSNKALPLP

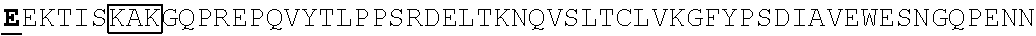

YKTTPPVLDSDGSFFLYSKLTVDKSRWQQGNVFSCSVMHEALHNHYTQKSLSLSPGK.
SEQ ID NO: 7 is an amino acid sequence of a modified mFc region of human IgG1 and
comprises an N-terminus of SEQ ID NO: 1 (underlined) (EV-Fc-201; aa 216-447):
EPKSGDKTHTSPPGPAPELLGGPSVFCFPPKPKDTLMISRTPEVTCVVVDVSHEDPEVKF
NWYVDGVEVHNAKTKPREEQYNSTYRVVSVLTVLHQDWLNGKEPKCKVSNKALPAPI

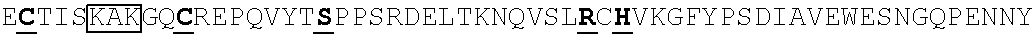

KTTKPVLDSDGSFFLYSKLTVDKSRWQQGNVFSCSVMHECLHNHYTQKSLSLSPGK.
SEQ ID NO: 8 is an amino acid sequence of a modified mFc region of human IgG1
hereof and comprises an N-terminus of SEQ ID NO: 1 (underlined) (Ev-Fc-203; aa 216-447):
EPKSGDKTHTSPPGPAPELLGGPDVFCFPPKPKDTLMISRTPEVTCVVVDVSHEDPEVKF
NWYVDGVEVHNAKTKPREEQYNSTYRVVSVLTVLHQDWLNGKEYKCKVSNKALPLP

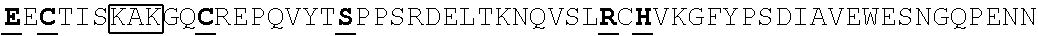

YKTTKPVLDSDGSFFLYSKLTVDKSRWQQGNVFSCSVMHECLHNHYTQKSLSLSPGK.
SEQ ID NO: 9 is an amino acid sequence of a modified mFc region of human IgG1 hereof
and comprises an N-terminus of SEQ ID NO: 1 (underlined) (Ev-Fc-203; aa 216-447):
EPKSGDKTHTSPPGPAPELLAGPDVFCFPPKPKDTLMISRTPEVTCVVVDVSHEDPEVKF
NWYVDGVEVHNAKTKPREEQYNSTYRVVSVLTVLHQDWLNGKEYKCKVSNKALPLP

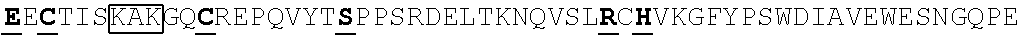

NNYKTTKPVLDSDGSFFLYSKLTVDKSRWQQGNVFSCSVMHECLHNHYTQKSLSLSPG
K.
SEQ ID NO: 10 is an amino acid sequence of SEQ ID NO: 7 without P343C and A431C
mutations (Ev-Fc-301; aa 216-447):
EPKSGDKTHTSPPGPAPELLGGPSVFCFPPKPKDTLMISRTPEVTCVVVDVSHEDPEVKF
NWYVDGVEVHNAKTKPREEQYNSTYRVVSVLTVLHQDWLNGKEYKCKVSNKALPAP

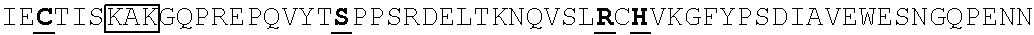

YKTTKPVLDSDGSFFLYSKLTVDKSRWQQGNVFSCSVMHEALHNHYTQKSLSLSPGK.
SEQ ID NO: 11 is an amino acid sequence of SEQ ID NO: 8 without P343C and A431C
mutations (Ev-Fc-302; aa 216-447):
EPKSGDKTHTSPPGPAPELLGGPDVFCFPPKPKDTLMISRTPEVTCVVVDVSHEDPEVKF
NWYVDGVEVHNAKTKPREEQYNSTYRVVSVLTVLHQDWLNGKEYKCKVSNKALPLP

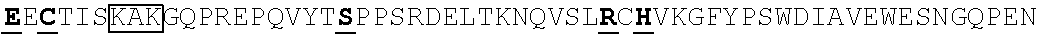

NYKTTKPVLDSDGSFFLYSKLTVDKSRWQQGNVFSCSVMHEALHNHYTQKSLSLSPG
K.
SEQ ID NO: 12 is an amino acid sequence of SEQ ID NO: 9 without P343C and A431C
mutations (Ev-Fc-303; aa 216-447):
EPKSGDKTHTSPPGPAPELLAGPDVFCFPPKPKDTLMISRTPEVTCVVVDVSHEDPEVKF
NWYVDGVEVHNAKTKPREEQYNSTYRVVSVLTVLHQDWLNGKEYKCKVSNKALPLP

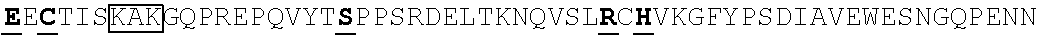

YKTTKPVLDSDGSFFLYSKLTVDKSRWQQGNVFSCSVMHEALHNHYTQKSLSLSPGK.
SEQ ID NO: 13 is an amino acid sequence of a modified mFc region of human IgG1
hereof with 14-aa extension at C-terminus (SEQ ID NO: 3) to enhance binding with FcRn and an
N-terminus of SEQ ID NO: 1 (Ev-Fc-401; aa 216-340):
EPKSGDKTHTSPPGPAPELLGGPSVFCFPPKPKDTLMISRTPEVTCVVVDVSHEDPEVKF
NWYVDGVEVHNAKTKPREEQYNSTYRVVSVLTVLHQDWLNGKEPKCKVSNKALPAPI

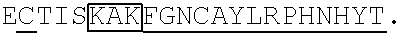

SEQ ID NO: 14 is an amino acid sequence of a modified mFc region of human IgG1
hereof with 14-aa extension at C-terminus (SEQ ID NO: 3) to enhance binding with FcRn and an
N-terminus of SEQ ID NO: 1 (Ev-Fc-402; aa 216-340):
EPKSGDKTHTSPPGPAPELLGGPDVFCFPPKPKDTLMISRTPEVTCVVVDVSHEDPEVKF
NWYVDGVEVHNAKTKPREEQYNSTYRVVSVLTVLHQDWLNGKEYKCKVSNKALPLP

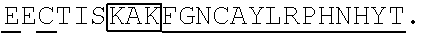

SEQ ID NO: 15 is an amino acid sequence of a modified mFc region of human IgG1
hereof with 14-aa extension at the C-terminus (SEQ ID NO: 3) to enhance binding with FcRn and
an N-terminus comprising SEQ ID NO: 1 (Ev-Fc-403; aa 216-340):
EPKSGDKTHTSPPGPAPELLAGPDVFCFPPKPKDTLMISRTPEVTCVVVDVSHEDPEVKF
NWYVDGVEVHNAKTKPREEQYNSTYRVVSVLTVLHQDWLNGKEYKCKVSNKALPLP

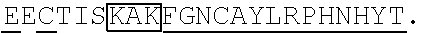

SEQ ID NO: 16 is an amino acid sequence of a wild-type Fc region of human IgG1,
wherein SEQ ID NO: 1 is underlined at the N-terminus (WT-Fc-C_{H}2):
EPKSGDKTHTSPPGPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAP
IEKTISKAKFGNCAYLRPHNHYT.
SEQ ID NO: 17 is a nucleic acid sequence that encodes the wild-type amino acids of SEQ
ID NO: 4:
GAGCCCAAGAGCGGCGACAAGACCCACACCAGCCCCCCCGGCCCCGCCCCCGAGCT
GCTGGGCGGCCCCAGCGTGTTCCTGTTCCCCCCCAAGCCCAAGGACACCCTGATGAT
CAGCAGGACCCCCGAGGTGACCTGCGTGGTGGTGGACGTGAGCCACGAGGACCCCG
AGGTGAAGTTCAACTGGTACGTGGACGGCGTGGAGGTGCACAACGCCAAGACCAA
GCCCAGGGAGGAGCAGTACAACAGCACCTACAGGGTGGTGAGCGTGCTGACCGTG
CTGCACCAGGACTGGCTGAACGGCAAGGAGTACAAGTGCAAGGTGAGCAACAAGG
CCCTGCCCGCCCCCATCGAGAAGACCATCAGCAAGGCCAAGGGCCAGCCCAGGGA
GCCCCAGGTGTACACCCTGCCCCCCAGCAGGGACGAGCTGACCAAGAACCAGGTGA
GCCTGACCTGCCTGGTGAAGGGCTTCTACCCCAGCGACATCGCCGTGGAGTGGGAG
AGCAACGGCCAGCCCGAGAACAACTACAAGACCACCCCCCCCGTGCTGGACAGCG
ACGGCAGCTTCTTCCTGTACAGCAAGCTGACCGTGGACAAGAGCAGGTGGCAGCAG
GGCAACGTGTTCAGCTGCAGCGTGATGCACGAGGCCCTGCACAACCACTACACCCA
GAAGAGCCTGAGCCTGAGCCCCGGCAAG.
SEQ ID NO: 18 is a nucleic acid sequence that encodes the amino acids of SEQ ID NO:
5:
GAGCCCAAGAGCGGCGACAAGACCCACACCAGCCCCCCCGGCCCCGCCCCCGAGCT
GCTGGCCGGCCCCGACGTGTTCCTGTTCCCCCCCAAGCCCAAGGACACCCTGATGAT
CAGCAGGACCCCCGAGGTGACCTGCGTGGTGGTGGACGTGAGCCACGAGGACCCCG
AGGTGAAGTTCAACTGGTACGTGGACGGCGTGGAGGTGCACAACGCCAAGACCAA
GCCCAGGGAGGAGCAGTACAACAGCACCTACAGGGTGGTGAGCGTGCTGACCGTG
CTGCACCAGGACTGGCTGAACGGCAAGGAGTACAAGTGCAAGGTGAGCAACAAGG
CCCTGCCCCTGCCCGAGGAGAAGACCATCAGCAAGGCCAAGGGCCAGCCCAGGGA
GCCCCAGGTGTACACCCTGCCCCCCAGCAGGGACGAGCTGACCAAGAACCAGGTGA
GCCTGACCTGCCTGGTGAAGGGCTTCTACCCCAGCGACATCGCCGTGGAGTGGGAG
AGCAACGGCCAGCCCGAGAACAACTACAAGACCACCCCCCCCGTGCTGGACAGCG
ACGGCAGCTTCTTCCTGTACAGCAAGCTGACCGTGGACAAGAGCAGGTGGCAGCAG
GGCAACGTGTTCAGCTGCAGCGTGATGCACGAGGCCCTGCACAACCACTACACCCA
GAAGAGCCTGAGCCTGAGCCCCGGCAAG.
SEQ ID NO: 19 is a nucleic acid sequence that encodes the amino acids of SEQ ID NO:
6:
GAGCCCAAGAGCGGCGACAAGACCCACACCAGCCCCCCCGGCCCCGCCCCCGAGCT
GCTGGGCGGCCCCGACGTGTTCCTGTTCCCCCCCAAGCCCAAGGACACCCTGATGAT
CAGCAGGACCCCCGAGGTGACCTGCGTGGTGGTGGACGTGAGCCACGAGGACCCCG
AGGTGAAGTTCAACTGGTACGTGGACGGCGTGGAGGTGCACAACGCCAAGACCAA
GCCCAGGGAGGAGCAGTACAACAGCACCTACAGGGTGGTGAGCGTGCTGACCGTG
CTGCACCAGGACTGGCTGAACGGCAAGGAGTACAAGTGCAAGGTGAGCAACAAGG
CCCTGCCCCTGCCCGAGGAGAAGACCATCAGCAAGGCCAAGGGCCAGCCCAGGGA
GCCCCAGGTGTACACCCTGCCCCCCAGCAGGGACGAGCTGACCAAGAACCAGGTGA
GCCTGACCTGCCTGGTGAAGGGCTTCTACCCCAGCGACATCGCCGTGGAGTGGGAG
AGCAACGGCCAGCCCGAGAACAACTACAAGACCACCCCCCCCGTGCTGGACAGCG
ACGGCAGCTTCTTCCTGTACAGCAAGCTGACCGTGGACAAGAGCAGGTGGCAGCAG
GGCAACGTGTTCAGCTGCAGCGTGATGCACGAGGCCCTGCACAACCACTACACCCA
GAAGAGCCTGAGCCTGAGCCCCGGCAAG.

### SUMMARY

A monomeric, fragment crystallizable domain (mFc) fragment of human immunoglobulin G1 (IgG1) is provided. The mFc fragment can (i) have a molecular weight of less than 40 kD, (ii) comprise (a) a C_{H}2 domain, (b) a C_{H}2 domain to the C terminus of which is a neonatal Fc receptor (FcRn) binding motif from a C_{H}3 domain is attached, (c) a C_{H}3 domain, or (d) both of a C_{H}2 domain and a C_{H}3 domain, (iii) bind to the FcRn and at least one Fcγ receptor, and (iv) comprise SEQ ID NO: 1 at an N-terminus of the fragment, optionally substituted with one or two conservative amino acid substitutions.

The FcRn binding motif from a C_{H}3 domain of the mFc fragment can be or comprise SEQ ID NO: 2. The mFc fragment can comprise SEQ ID NO: 3 at its C-terminus, which can be optionally substituted with one or two conservative amino acid substitutions.

The mFc fragment can comprise an IKAROS family zinc finger 1 (IKS) at amino acid positions corresponding to amino acids 338-340 (KAK) of SEQ ID NO: 4. The mFc fragment can comprise a deletion corresponding to amino acids 341-447 of SEQ ID NO: 4. The mFc fragment can comprise a serine at the amino acid position corresponding to amino acid 431 (C) of SEQ ID NO: 4.

The mFc fragment can comprise an amino acid sequence of SEQ ID NO: 5. The mFc fragment can comprise an amino acid sequence of SEQ ID NO: 6. The mFc fragment can comprise an amino acid sequence of SEQ ID NO: 7. The mFc fragment can comprise an anamino acid sequence of SEQ ID NO: 8. The mFc fragment can comprise an amino acid sequence of SEQ ID NO: 9. The mFc fragment can comprise an amino acid sequence of SEQ ID NO: 10. The mFc fragment can comprise an amino acid sequence of SEQ ID NO: 11. The mFc fragment can comprise an amino acid sequence of SEQ ID NO: 12. The mFc fragment can comprise an amino acid sequence of SEQ ID NO: 13. The mFc fragment can comprise an amino acid sequence of SEQ ID NO: 14. The mFc fragment can comprise an amino acid sequence of SEQ ID NO: 15.

The at least one Fcγ receptor can be FcγRI, FcγRIIa, FcγRIIb, and/or FcγRIII. The mFc fragment can comprise at least one mutation that increases affinity to at least one Fcγ receptor. In certain embodiments, the at least one mutation is at least one of S239D, A330L, and I332E.

The at least one Fcγ receptor can be FcγRIII.

The mFc fragment can comprise at least one mutation that increases affinity to the FcRn receptor comparative to wild-type. In certain embodiments, the at least one mutation that increases affinity to the FcRn receptor is selected from the group consisting of H433K/N434F/Y436H and H433K/N434F.

The mFc fragment can comprise at least one mutation that increases in vivo stability of the fragment. In certain embodiments, the at least one mutation that increases in vivo stability of the fragment is at least one of L242C, K334C, P343C, and A431C.

The mFc fragment can comprise at least one mutation that decreases or eliminates aggregation of the fragment. In certain embodiments, the at least one mutation that decreases or eliminates aggregation of the fragment is at least one of L351S, T366R, L368H, and P395K.

The mFc fragment can comprise at least one site for conjugation. The at least one site for conjugation can be at least one of a cysteine, a serine, a lysine, a tyrosine, and an unnatural amino acid.

Conjugates are also provided. In certain embodiments, the conjugate has a structure of Formula I:

mFc-L-smL (Formula I)

or is a pharmaceutically acceptable salt thereof, wherein: mFc any of the mFc fragments described herein; smL is a radical of a small molecule ligand that binds to neuraminidase (NA) or hemagglutinin (HA) on influenza virus or an influenza virus-infected cell; and L is a linker that is covalently bound to smL and mFc.

The smL can be a radical of a NA inhibitor or an HA inhibitor. The smL can be a radical of zanamivir. The smL can be a radical of oseltamivir, peramivir, or laninamivir.

The mFc can comprise at least one cysteine, lysine, and/or tyrosine via which L is covalently bound to mFc.

In certain embodiments, L is covalently bound to mFc via an unnatural amino acid. L can further comprise a spacer. L can be non-cleavable. L can increase the water-solubility of the conjugate. L can comprise one or more of an amino acid, a polyethylene glycol (PEG) monomer, a PEG oligomer, a PEG polymer, or a combination of two or more of the foregoing. L can comprise an oligomer of peptidoglycans, glycans, anions, or a combination of two or more of the foregoing.

A composition comprising any of the conjugates described herein is also provided, wherein the composition comprises the conjugate and a pharmaceutically acceptable carrier.

Methods for delivering an immune effector to an influenza virus or a cell infected with influenza virus in a subject are provided. In certain embodiments, the method for delivering an immune effector to an influenza virus or a cell infected with influenza virus in a subject comprises administering to the subject an effective amount of a conjugate hereof or a composition comprising the conjugate and a pharmaceutically acceptable carrier.

The immune effector can be any mFc fragment described herein.

The conjugate or composition can be administered intravascularly, orally, or intranasally.

Methods for treating a subject having a viral infection or a cell infected with a virus are also provided. In certain embodiments, the method for treating a subject having a viral infection or a cell infected with a virus comprises administering to the subject an effective amount of a conjugate described herein or a composition comprising the conjugate and a pharmaceutically acceptable carrier.

The immune effector can be any mFc fragment described herein. The conjugate or composition can be administered intravascularly, orally, or intranasally. The conjugate or composition can be administered to the subject in a single dose. The conjugate or composition can be administered to the subject in more than one dose. The virus can be an Influenza virus. The virus can be an Influenza A virus.

### BRIEF DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1 shows a sequence alignment of certain C_{H}2-C_{H}3 mutations of the monomeric Fc (mFc) fragments hereof as compared to a wild-type (WT) Fc fragment.

FIG. 2 shows the 3D modeled structure of a wild-type C_{H}2-C_{H}3 fragment.

FIG. 3 shows a sequence alignment of certain C_{H}2 mutations of the mFc fragments hereof as compared to a WT Fc fragment.

FIG. 4 shows the 3D modeled structure of a wild-type C_{H}2 fragment.

FIG. 5 shows a schematic example of the thiol specific drug conjugation (1:4=ratio of zanamivir conjugates to each Fc fragment, resulting in a conjugate that contains a single Fc conjugated to 4 zanamivir molecules).

FIG. 6 shows surrogate antibody-dependent cell-mediated cytotoxicity (ADCC) assay data.

FIG. 7 shows half maximal effective concentrations of the drug conjugates.

FIG. 8 shows treatment of influenza A/H1N1/PR8-infected mice with a single, intravenous dose of zan-Fc conjugate 48 hours post-infection (hpi).

FIG. 9A shows treatment of influenza A/H1N1/PR8-infected mice with different conjugates beginning 48 hpi. Single doses of conjugates were administered except five doses of zan-folates were administered.

FIG. 9B shows treatment of influenza A/H1N1/PR8-infected mice with different conjugates beginning 72 hpi. Single doses of conjugates were administered except five doses of zan-folates were administered.

FIG. 9C shows treatment of influenza A/H1N1/PR8-infected mice with different conjugates beginning 96 hpi. Single doses of conjugates were administered except five doses of zan-folates were administered.

While the present disclosure is susceptible to various modifications and alternative forms, exemplary embodiments thereof are shown by way of example in the drawings and are herein described in detail.

### DETAILED DESCRIPTION

While the concepts of the present disclosure are illustrated and described in detail in the description herein, results in the description are to be considered as exemplary and not restrictive in character; it being understood that only the illustrative embodiments are shown and described and that all changes and modifications that come within the spirit of the disclosure are desired to be protected.

The present disclosure provides materials and methods for delivering an immune effector to the surface of a virus or a virus-infected cell in a subject. More specifically, the present disclosure is directed to the design of Fc (fragment crystallizable domain) fragments, in particular monomeric C_{H}2 and monomeric C_{H}2-C_{H}3 fragments. The monomeric Fc (mFc) fragments hereof are polypeptides of human immunoglobulin G1 (IgG1), which are stable, effectively engage immune cells, and effectively penetrate tissues, including solid tumors. Conjugates are also provided that comprise the Fc monomeric fragments, as are pharmaceutical compositions. Methods of making and using such molecules, conjugates, and compositions are also provided.

Monoclonal antibodies (mAbs) are widely used for therapeutic applications. While mAbs represent the largest class of biological drugs, their large size results in poor tissue penetration. Consequently, various small antibody formats have been developed, such as fragment antigen-binding regions (Fabs), variable domains (Fv), single-chain variable fragments (scFv), variable region of the heavy chain (V_{H}) and nanobodies (VHHs). While the small antibody formats allow for better tissue penetration (comparative to mAbs), they have relatively short half-lives.

The wild-type Fc is homodimeric in nature and this feature is driven by the strong, high-affinity interaction that exists between the two C_{H}3 domains. Although the term “Fc” is typically thought of as a homodimer of polypeptides, the term as used herein, will also include monomeric polypeptides which comprise a sequence of amino acids corresponding to the Fc portion of the heavy chain (e.g., containing a C_{H}2 and C_{H}3 domain).

### mFc Fragments of Human IgG1

In view of the above, an mFc fragment of human immunoglobulin G1 (IgG1) is provided. The mFc fragment is a polypeptide and, more specifically, mFc fragments of human IgG1 are portions of the antibody molecule that consist of the constant fragment of the heavy chain (Fc) region of a single IgG1 molecule. The Fc region of IgG1 is responsible for many of the antibody's effector functions, such as binding to Fc receptors on immune cells, activating complement cascade, and facilitating antibody-dependent cell-mediated cytotoxicity (ADCC). The Fc region is also involved in the regulation of the immune response and the transport of the IgG1 across cell membranes.

In certain embodiments, the mFc fragment has a molecular weight of less than about 40 kDa (e.g., less than 40 kDa). The mFc fragment can have a molecular weight of at or about 25 kDa (e.g., 25 kDa). The mFc fragment can have a molecular weight of at or about 26 kDa (e.g., 26 kDa). The mFc fragment can have a molecular weight of at or about at or about 30 kDa (e.g., 30 kDa). The mFc fragment can have a molecular weight of at or about 35 kDa (e.g., 35 kDa).

The mFc fragment can comprise an N-terminus and a C-terminus. The N-terminus of the mFc fragment can comprise SEQ ID NO: 1, optionally substituted with one or two conservative amino acid substitutions. Additionally or alternatively, the C-terminus of the mFc fragment can comprise SEQ ID NO: 3, optionally substituted with one or two conservative amino acid substitutions.

As used herein, “conservative amino acid substitution” or, simply, “conservative variations” of a particular sequence refers to the replacement of one amino acid, or series of amino acids, with amino acid sequences that do not substantially affect or decrease the activity of the peptide/polypeptide/protein. One of skill will recognize that individual substitutions, deletions, or additions which alter, add, or delete a single amino acid or a percentage of amino acids in an encoded sequence result in “conservative variations” where the alterations result in the deletion of an amino acid, addition of an amino acid, or substitution of an amino acid with a chemically similar amino acid. It is understood that the addition of sequences that do not alter the encoded activity of a polypeptide, such as the addition of a non-functional sequence, is a conservative variation of the basic amino acid. Conservative substitutions generally maintain the structure of the polypeptide backbone in the area of the substitution, the charge/hydrophobicity at the target site, and/or the bulk of the side chain.

Conservative substitution tables providing functionally similar amino acids are well known in the art and non-limiting examples of conservative amino acid substitutions are provided in Table 1. Thus, “conservative amino acid substitutions” of a listed polypeptide sequence (e.g., SEQ ID NOs: 1-15) include substitutions of a percentage, typically less than 10%, of the amino acids of the polypeptide sequence, with a conservatively selected amino acid of the same conservative substitution group. Conservative amino acid substitutions in one or a few amino acids in an amino acid sequence are substituted with different amino acids with highly similar properties of each disclosed sequence are a feature of the polypeptides provided herein.

TABLE 1
Conservative amino acid substitutions

| Amino acid | Conservative Substitutions |
|---|---|
| Ser, S | Thr, Gly, Asn |
| Arg, R | His, Lys, Glu, Gln |
| Leu, L | Ile, Met, Phe, Val, Tyr |
| Pro, P | Ala, Thr, Gly |
| Thr, T | Pro, Ser, Ala, Gly, His, Gln |
| Ala, A | Pro, Gly, Thr |
| Val, V | Met, Ile, Tyr, Phe, Leu |
| Gly, G | Ala, Thr, Pro, Ser |
| Ile, I | Met, Leu, Phe, Val, Tyr |
| Phe, F | Met, Tyr, Ile, Leu, Trp, Val |
| Tyr, Y | Phe, Trp, Met, Ile, Val, Leu |
| Cys, C | Ser, Thr, Met |
| His, H | Gln, Arg, Lys, Glu, Thr |
| Gln, Q | Glu, His, Lys, Asn, Thr, Arg |
| Asn, N | Asp, Ser, Gln |
| Lys, K | Arg, Glu, Gln, His |
| Asp, D | Asn, Glu, Gln |
| Glu, E | Gln, Asp, Lys, Asn, His, Arg |
| Met, M | Ile, Leu, Phe, Val |

The mFc fragment can comprise a C_{H}2 domain. The mFc fragment can comprise a C_{H}3 domain. The mFc fragment can comprise both a C_{H}2 domain and a C_{H}3 domain. In certain embodiments, the mFc fragment comprises a C_{H}2 domain to the C-terminus of which a neonatal Fc receptor (FcRn) binding motif from a C_{H}3 domain is attached. The FcRn binding motif from a C_{H}3 domain can be or comprise SEQ ID NO: 2.

The mFc fragment can comprise a C_{H}2 domain and/or C_{H}3 domain variant. A “variant” of a polypeptide comprises an amino acid sequence wherein one or more amino acid residues are inserted into, deleted from, and/or substituted into the amino acid sequence relative to another polypeptide sequence. Variants hereof can include those comprising a variant C_{H}2 or C_{H}3 domain. In certain embodiments, a variant comprises one or more mutations that, when present in an Fc molecule, increase affinity for the polypeptide to one or more Fcγ receptors and/or FcRns.

In certain embodiments, the mFc fragment comprises IKAROS family zinc finger 1 (IKS) at amino acid positions corresponding to amino acids 338-340 (KAK) of SEQ ID NO: 4. Introduction of this mutation, for example, can further increase the stability and aggregation resistance of the mFc fragment. In certain embodiments, the mFc fragment comprises a deletion corresponding to amino acids 341-447 of SEQ ID NO: 4. The mFc fragment can comprise a serine at the amino acid position corresponding to amino acid 431 (C) of SEQ ID NO: 4.

The mFc fragment can interact/bind to the FcRn and at least one Fcγ receptor (e.g., a Fcγ receptor on the surface of an immune cell in a subject and/or the FcRn on the surface of an endothelial cell or macrophage of a subject). The Fcγ receptor can be FcγRI (CD64), FcγRIIa (CD32), FcγRIIb (CD32B), and/or FcγRIII (CD16) (e.g., FcγRIIIa and/or FcγRIIIb). Binding to FcγRI can help phagocytosis by activated macrophages. Binding to FcγR3 can help kill natural killer (NK) cells.

In certain embodiments, the mFc fragment of IgG1 has (i) has a molecular weight of less than 40 kD; (ii) comprises (a) a C_{H}2 domain, (b) a C_{H}2 domain to the C terminus of which a FcRn binding motif from a C_{H}3 domain is attached, (c) a C_{H}3 domain, or (d) both of a C_{H}2 domain and a C_{H}3 domain; (iii) binds to the FcRn and at least one Fcγ receptor; and (iv) comprises at its N-terminus SEQ ID NO: 1, which is optionally substituted with one or two conservative amino acid substitutions. The FcRn binding motif from a C_{H}3 domain can have the amino acid sequence SEQ ID NO: 2. The mFc fragment can comprise at its C-terminus SEQ ID NO: 3, which is optionally substituted with one or two conservative amino acid substitutions (see, e.g., Table 1). The mFc fragment can comprise IKS at amino acid positions corresponding to amino acids 338-340 (KAK) of SEQ ID NO: 4. The mFc fragment can comprise a deletion corresponding to amino acids 341-447 of SEQ ID NO: 4. The mFc fragment can comprise a serine at the amino acid position corresponding to amino acid 431 (C) of SEQ ID NO: 4. The Fcγ receptor can be FcγRI, FcγRIIa, FcγRIIb, and/or FcγRIII. Binding to FcγRI can help phagocytosis by activated macrophages. Binding to FcγR3 can help kill NK cells.

The mFc fragment can comprise the amino acid sequence of SEQ ID NO: 5. The mFc can comprise the amino acid sequence of SEQ ID NO: 6, 7, 8, 9, 10, 11, 12, 13, 14 or 15. The mFc can have at least 60% sequence identity to SEQ ID NO: 5, 6, 7, 8, 9, 10, 11, 12, 13, 14, or 15. In a particular embodiment, the mFc comprises at least 65%, 70%, 75%, 80%, 85%, 88%, 90%, 92%, 95%, 98%, or at least 99% identity to SEQ ID NO: 5, 6, 7, 8, 9, 10, 11, 12, 13, 14, or 15.

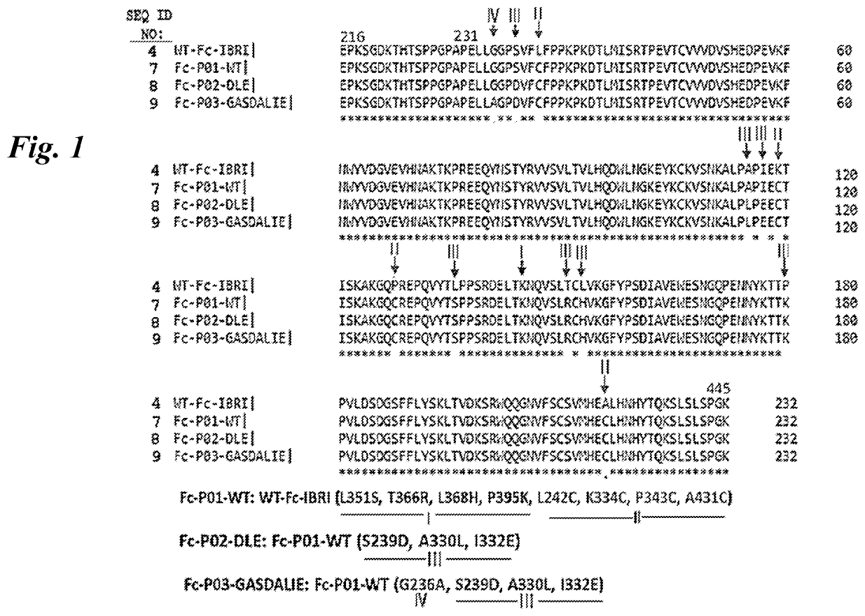

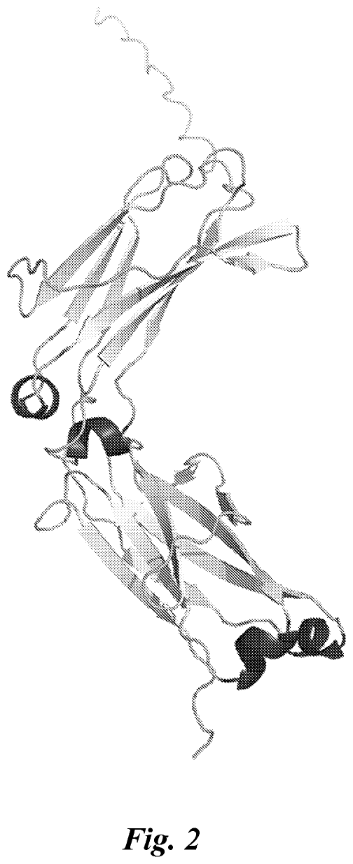

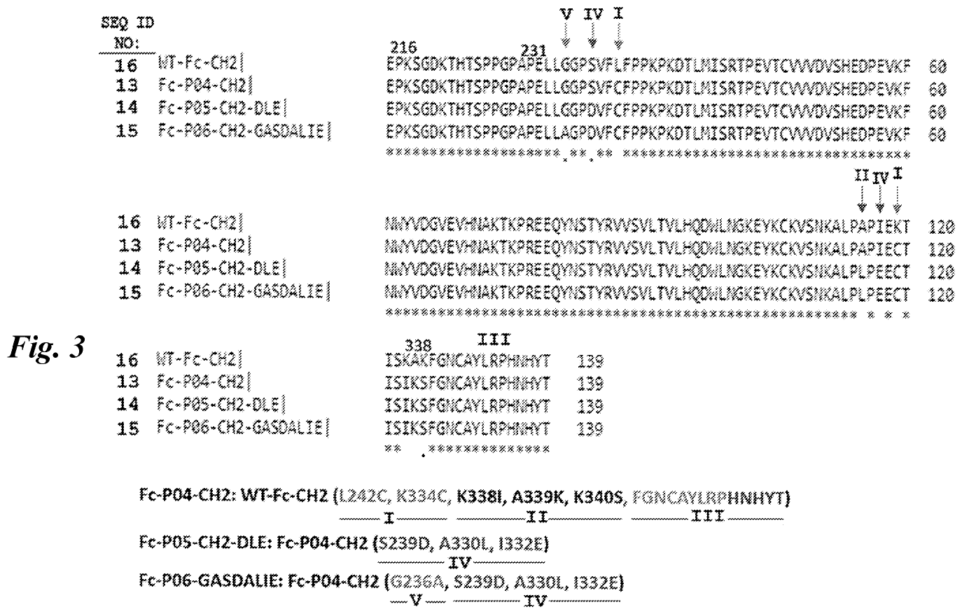

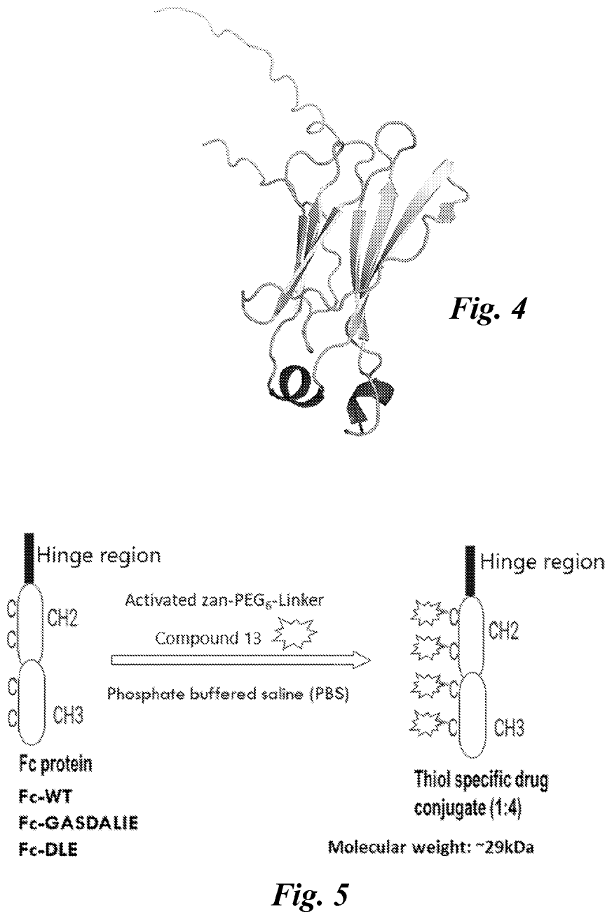

“Sequence identity” or “percent identity” herein describes the extent to which two nucleotide or amino acid sequences are invariant in an alignment of sequences. “Sequence alignment” means the process of lining up two or more sequences to achieve maximal levels of identity (and, in the case of amino acid sequences, conservation) for the purpose of assessing the degree of similarity. As shown in FIG. 1, an alignment of sequences is created by manually aligning at least two sequences, for example, a stated sequence as a reference and another sequence, to produce the highest number of matching elements (e.g., individual nucleotides or amino acids) while allowing for the introduction of gaps into either sequence. An “identity fraction” for a sequence aligned with a reference sequence is the number of matching elements, divided by the full length of the reference sequence, not including gaps introduced by the alignment process into the reference sequence. “Percent identity” as used herein is the identity fraction times 100.

The mFc fragment can comprise at least one mutation that increases affinity to at least one Fcγ receptor (e.g., comparative to wild-type or the mFc fragment without the mutation), at least one mutation that increases affinity to the FcRn receptor (e.g., comparative to wild-type or the mFc fragment without the mutation), at least one mutation that increases in vivo stability of the fragment (e.g., comparative to wild-type or the mFc fragment without the mutation), and/or at least one mutation that decreases or eliminates aggregation of the fragment. While these mutations can comprise one or more conservative amino acid substitutions (see, e.g., Table 1 and described above), such mutations also can include non-conservative amino acid substitutions. Examples of non-conservative amino acid substitutions include substitution of a hydrophilic residue (e.g., Ser or Thr) with a hydrophobic residue (e.g., Leu, Ile, Phe, Val or Ala) or vice versa, substitution of a Cys or Pro with any other residue or vice versa, substitution of an amino acid having an electropositive side chain (e.g., Lys, Arg, or His) with an amino acid having an electronegative side chain (e.g., Glu or Asp) or vice versa, or substitution of an amino acid having a bulky side chain (e.g., Phe) with an amino acid not having a bulky side chain (e.g., Gly).

The mFc fragment can comprise at least one mutation that increases affinity to at least one FcRn receptor. The at least one mutation that increases affinity to the FcRn receptor can be selected from the group consisting of a triple mutation of H433K/N434F/Y436H (i.e., changing histidine at position 433 to lysine, asparagine at position 434 to phenylalanine, and tyrosine at position 436 to histidine in the FcRn receptor binding motif) and a double mutation of H433K/N434F (i.e., changing histidine at psotion 433 to lysine and asparagine at position 434 to phenylalanine in the FcRn receptor binding motif). Dall'Acqua et al., Increasing the affinity of a human IgG1 for the neonatal Fc receptor: biological consequences, J Immunology 169 (9): 5171-5180 (2002); Lee et al., An engineered human Fc domain that behaves like a pH-toggle switch for ultra-long circulation persistence, Nature Communications 10:5031 (2019).

The mFc fragment can comprise at least one mutation that increases affinity to at least one Fcγ receptor (e.g., FcγRIII). The at least one mutation that increases affinity to at least one Fcγ receptor can be at least one of S239D, A330L, and 1332E.

The mFc fragment can comprise at least one mutation that increases in vivo stability of the fragment. The at least one mutation that increases in vivo stability of the fragment can be at least one of L242C, K334C, P343C, and A431C. The mFc fragment can comprise at least one mutation that decreases or eliminates aggregation of the fragment (e.g., in vivo). The at least one mutation that decreases or eliminates aggregation of the fragment can be at least one of L351S, T366R, L368H, and P395K. The at least one mutation that decreases or eliminates aggregation of the fragment can be a mutation that increase the thermal stability of the fragment and/or reduce exposure to hydrophobic patches that promote protein-protein interactions.

The mFc fragment can comprise at least one site for conjugation. The at least one site for conjugation can be at least one of a cysteine, a serine, a lysine, a tyrosine, and an unnatural amino acid (UAA). If the cysteine at position 431 is mutated to serine, the only naturally occurring cystine available for site-specific conjugation is C343. Y296 is the only predicted solvent-exposed tyrosine residue of the C_{H}2-C_{H}3 and C_{H}2 fragments, which can be utilized for site-specific conjugation.

UAAs also can be incorporated into the fragments for site-specification conjugation. For example, an azido lysine can be incorporated at K246 position of C_{H}2 and K360 position of C_{H}2-C_{H}3 for site-specific conjugation.

The mFc fragment can be glycosylated or unglycosylated. For example, a recombinant mFc fragment can be expressed in a mammalian that glycosylates the expressed mFc fragment.

In general, the IgG1 C_{H}2, C_{H}3 and C_{H}2-C_{H}3 domain wild-type and mutants can be synthesized or cloned. The sequences can be synthesized by any suitable method known in the art, such as the phosphotriester method, the phosphodiester method, the diethylphosphoramidite method, the solid phase phosphoramidite triester method, and the solid support method of U.S. Pat. No. 4,458,066. Narang et al., Improved phosphotriester method for the synthesis of gene fragments, Methods in Enzymology 68:90-99 (1979); Brown et al., Chemical synthesis and cloning of a tyrosine tRNA gene, Methods in Enzymology 68:109-151 (1979); Beaucage et al., Deoxynucleoside phosphoramidites—a new class of key intermediates for deoxypolynucleotide synthesis, Tetrahedron Letters 22:1859-1862 (1981).

The sequences can be cloned by any suitable method known in the art. See, e.g., Molecular Cloning: A Laboratory Manual, Green et al., 4^{th} ed. (2012); Berger & Kimmel, Methods in Enzymology, vol. 152: Guide to Molecular Cloning Techniques, 1^{st} ed. (1987); Short Protocols in Molecular Biology, Ausubel et al., eds. 4^{th} ed. (1999). In some embodiments, nucleic acid molecules which encode a polypeptide (e.g., mFc fragment) set forth in any of SEQ ID NOs: 6, 7, 8, 9, 10, 11, 12, 13, 14, or 15 are provided. These nucleic acid molecules can be isolated using standard molecular biology techniques and the sequence information provided herein. Nucleic acids can be prepared by amplification, such as polymerase chain reaction (PCR), ligase chain reaction (LCR), transcription-based amplification system (AS), or the self-sustained sequence replication system (3SR). Furthermore, oligonucleotides corresponding to nucleotide sequences can be prepared by standard synthetic techniques.

The IgG1 C_{H}2, C_{H}3 and C_{H}2-C_{H}3 domain wild-type and mutants then can be subcloned into a vector in operable linkage with a regulatory sequence such as a promoter. Expression vectors include, but are not limited to, plasmids, phages, viruses, adenoviruses, bacterial artificial chromosomes, yeast artificial chromosomes, and other vehicles suitable for expressing a polypeptide. Any vector that transduces genetic material into a cell and, if replication is desired, which is replicable and viable in the relevant host can be used.

The term “vector” or “expression vector” means the vehicle by which a deoxyribonucleic acid (DNA) or ribonucleic acid (RNA) sequence (e.g., a foreign gene) can be introduced into a host cell so as to transform the host and promote expression (e.g., transcription and translation) of the introduced sequence. Vectors typically comprise the DNA of a transmissible agent, into which foreign DNA encoding a protein (e.g., a mFc fragment thereof) is inserted by restriction enzyme technology or the like. A common type of vector is a plasmid, which generally is a self-contained molecule of double-stranded DNA that can readily accept additional (foreign) DNA and which can be readily introduced into a suitable host cell. A large number of vectors, including plasmid and fungal vectors, have been described for replication and/or expression in a variety of eukaryotic and prokaryotic hosts. The terms “express” and “expression” mean allowing or causing the information in a gene or DNA sequence to become manifest, for example by producing a protein by activating the cellular functions involved in transcription and translation of a corresponding gene or DNA sequence. A DNA sequence is expressed in or by a cell to form an “expression product” such as a protein or polypeptide (e.g., a mFc fragment hereof). The expression product itself, e.g., the resulting protein, can also be said to be “expressed” by the cell. A polypeptide can be expressed recombinantly, for example, when it is expressed or produced in a foreign cell under the control of a foreign or native promoter, or in a native host cell under the control of a foreign promoter.

The regulatory sequence can comprise a promoter operably linked to the sequence. The promoter can be constitutive or inducible. The promoter can optionally include distal elements, such as enhancer or repressor elements. Other elements include those that control promoter-dependent gene expression by cell or tissue type or induction by an external agent or signal. An example of a promoter is the cytomegalovirus (CMV) promoter. Large numbers of suitable vectors and promoters are known to those of skill in the art and are commercially available.

The vector can be introduced into a host cell for replication and expression of the targeted polypeptide or protein. Examples of host cells include microbial, yeast, insect, and mammalian organisms and cell lines (e.g., COS, CHO, HeLa and myeloma). The vector can be introduced using any suitable method known in the art. Examples of methods include transformation, transfection, microinjection, electroporation, and liposomes.

Recombinantly expressed polypeptides can be carried out by conventional means, such as preparative chromatography and immunological separations. Purification can be accomplished using standard procedures, such as ammonium sulfate precipitation, affinity columns, column chromatography, and the like.

Alternatively, such polypeptides can be synthesized, in whole or in part, using standard peptide synthesis. See, e.g., Barany & Merrifield, The Peptides: Analysis, Synthesis, Biology. Vol. 2: Special methods in Peptide Synthesis, Part A. pp. 3-284; Merrifield et al., JACS 85:2149-2156 (1963); and Stewart et al., Solid Phase Peptide Synthesis, 2^{nd} ed., Pierce Chem. Co., Rockford, IL (1984).

### Conjugates

A conjugate of Formula I is also provided:

mFc-L-smL (Formula I),

wherein mFc is any of the monomeric Fc fragments described above, L is a linker, and smL is a radical of a small molecule ligand that binds to an enveloped virus or a cell infected with an enveloped virus.

mFc can be any of the mFc fragments described herein. In certain embodiments, the mFc is of human IgG1, has a molecular weight of less than about 40 kDa (e.g., less than 40 kDa) and comprises (a) a C_{H}2 domain, (b) a C_{H}2 domain to the C terminus of which a FcRn binding motif from a C_{H}3 domain is attached, (c) a C_{H}3 domain, or (d) both of a C_{H}2 domain and a C_{H}3 domain. The mFc can bind to the FcRn and at least one Fcγ receptor and can further comprise SEQ ID NO: 1 at an N-terminus of the fragment, optionally substituted with one or two conservative amino acid substitutions. In certain embodiments, the mFc comprises the amino acid sequence of SEQ ID NO: 5, 6, 7, 8, 9, 10, 11, 12, 13, 14, or 15. In certain embodiments, the mFc comprises an amino acid sequence with at least 60% sequence identity to SEQ ID NO: 5, 6, 7, 8, 9, 10, 11, 12, 13, 14, or 15.

The smL is a radical of a small molecule ligand that binds to an enveloped virus or a cell infected with an enveloped virus. For example, the smL can have specificity for binding an envelope protein of a virus or a viral envelope protein on the surface of a virus-infected cell.

Influenza virus, for example, is an enveloped virus. All influenza subtypes are very similar in overall structure and composition; namely, a virus particle is 80-120 nanometers in diameter and has a viral envelope containing two main types of glycoproteins wrapped around a central core. The central core contains the viral RNA genome and other viral proteins that package and protect this RNA. Unusually for a virus, influenza's genome is not a single piece of nucleic acid but rather seven or eight pieces of segmented negative-sense RNA, each piece of which contains either one or two genes that code for a gene product (protein). For example, the influenza A genome contains 11 genes on eight pieces of RNA that encode for 11 proteins: hemagglutinin (HA), neuraminidase (NA), nucleoprotein (NP), M1, M2, NS1, NS2 (NEP: nuclear export protein), PA, PB1 (polymerase basic 1), PB1-F2, and PB2.

HA and NA are the two large glycoproteins on the outside of the viral particles. HA is a lectin that can mediate binding of the virus to target cells and entry of the viral genome into a target cell, while NA is typically involved in the release of progeny virus from infected cells by cleaving sugars that bind the mature viral particles. The target protein can be influenza NA or influenza HA.

In some embodiments of the conjugate hereof, the smL is a radical of a NA or HA inhibitor, which can be used to deliver the conjugate into a virus infected cell and/or virus replication sites (e.g., nose, throat, and lungs). This can allow for killing virus infected cells prior to the progeny virus release, hindering viral replication, and/or dampening the early cytokine storm induced by viral infection. In certain embodiments, the smL is a radical of oseltamivir, zanamivir, peramivir, or laninamivir. In certain embodiments, the smL is a radical of zanamivir.

In other embodiments, the smL can have specificity for binding a protein is selected from respiratory syncytial virus (RSV) fusion protein F, a coronavirus spike protein, and a hepatitis B virus (HBV) surface antigen or HBV core antigen.

The conjugate comprises a linker (L) that couples or otherwise connects or binds the mFc fragment to the radical of the small molecule ligand. As used herein, the term “linker” includes a chain of atoms that is bio-functionally adapted to form a chemical bond (e.g., a covalent bond) with the mFc and smL and connects two or more functional parts of a molecule to form a conjugate hereof. Illustratively, the chain of atoms can be selected from carbon (C), nitrogen (N), oxygen (O), sulfur(S), silicon (Si), and phosphorus (P). The chain of atoms can be selected from C, N, O, S, and P, C, N, O, and S. The chain of atoms can covalently connect different functional capabilities of the conjugate, such as the mFc fragment and the small molecule ligand radical. The linker can comprise a wide variety of links, such as in the range from about 2 to about 100 atoms in the contiguous backbone.

The linker can comprise a C₂-C₁₈ alkyl group, a peptide radical, or a peptidoglycan radical. In this context, the term “fragment” means a molecule that has been modified to allow linking in the conjugate either as monovalent linking, such as in the case of a mFc fragment or bivalent linking, such as in the case of the linker. The term “radical” means a chemical species that contains one or more unpaired electrons in its outer shell and is therefore reactive.

For example, the term “zanamivir radical” means that zanamivir has been chemically modified to allow it to be prepared as the small molecule ligand covalently bound to a linker. With respect to zanamivir, a proton, such as from an alcohol, is missing, thus creating an open valence on the corresponding oxygen, which is bound, in turn, to the linker. Typically, the alcohol oxygen closest to the dihydropyran ring of zanamivir forms a bond to linker. Similarly, mFc fragments are moieties based on the corresponding molecules which have been adapted for linking to the linker. With regards to the linker, the corresponding fragments and radicals have open valencies to allow for bonding to other linker moieties, mFc fragments, and/or small molecule ligands. For example, with respect to PEG, the term (—CH₂CH₂—O—)ₙ refers to a fragment that is bound on both sides to another moiety, such as another linker component, for example. The use of the term “fragment” does not require that from a synthetic perspective; the molecule it refers to can be made in the preparation of the conjugate. It is a description for moiety within the conjugate, regardless of how made.

In certain embodiments, the linker is covalently bound to the small molecule ligand and the mFc fragment. The mFc fragment can comprise at least one cysteine, lysine, and/or tyrosine via which the linker is covalently bound to the mFc fragment. The liner can be covalently bound to the mFc fragment via a UAA.

The linker can further comprise a spacer. In some embodiments, the spacer comprises a peptidoglycan or a sugar.

The linker can be non-cleavable. For example, the linker can comprise a slow-release linker (e.g., a non-hydrolyzable linker or a linker that is resistant to hydrolysis).

The linker can increase the water-solubility of the conjugate. For example, and without limitation, the linker can include one or more of hydrophilic functional groups such as —OH, —COOH, —NH₂, and —SO₃H, ionic functional groups such as —COO—, —SO₃, —NH₃⁺, and/or —N⁺(CH₃)₃, PEG, fatty acids or phosopholipids, and/or cyclodextrin-based linkers.

The linker can comprise one or more of an amino acid, a polyethylene glycol (PEG) monomer, a PEG oligomer, a PEG polymer, or a combination of two or more of the foregoing. The linker can comprise an oligomer of peptidoglycans, glycans, anions, or a combination of two or more of the foregoing.

Conjugates can be synthesized in accordance with methods known in art and exemplified herein.

### Salts

The conjugates can be presented as a pharmaceutically acceptable salt. Examples of acceptable salts include, without limitation, alkali metal (e.g., sodium, potassium, or lithium) or alkaline earth metal (e.g., calcium) salts; however, any salt that is generally non-toxic and effective when administered to the subject being treated is acceptable. Similarly, “pharmaceutically acceptable salt” refers to those salts with counter ions, which can be used in pharmaceuticals. More specifically with respect to the present disclosure, the terms “salts” and “pharmaceutically acceptable salts” as used herein refer to derivatives of the disclosed conjugates wherein the parent conjugate is modified by making acid or base salts thereof.

Such salts can include, without limitation, (1) acid addition salts, which can be obtained by reaction of the free base of the parent compound with inorganic acids, such as hydrochloric acid, hydrobromic acid, nitric acid, phosphoric acid, sulfuric acid, perchloric acid, and the like, or with organic acids, such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or (2) salts formed when an acidic proton present in the parent compound either is replaced by a metal ion, e.g., an alkali metal ion, an alkaline earth ion, or an aluminum ion, or coordinated with an organic base, such as ethanolamine, diethanolamine, triethanolamine, trimethamine, N-methylglucamine, and the like. Pharmaceutically acceptable salts are well-known to those skilled in the art, and any such pharmaceutically acceptable salts are contemplated.

Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent conjugate which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa. (1985).

Acceptable salts can be obtained using standard procedures known in the art, including (without limitation) reacting a sufficiently acidic compound with a suitable base affording a physiologically acceptable anion. Suitable acid addition salts are formed from acids that form non-toxic salts. Illustrative, albeit nonlimiting, examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts of the compounds can be formed from bases that form non-toxic salts. Illustrative, albeit nonlimiting, examples include arginine, benzathine, calcium, choline, diethylamine, diolamine, glycine, lysine, magnesium, meglumine, olamine, potassium, sodium, tromethamine and zinc salts. Hemi-salts of acids and bases, such as hemi-sulphate and hemi-calcium salts, also can be formed.

The conjugates hereof can be “deuterated,” meaning one or more hydrogen atoms can be replaced with deuterium. As deuterium and hydrogen have nearly the same physical properties, deuterium substitution is the smallest structural change that can be made. Deuteration is well known to those of ordinary skill in the art.

The conjugates, in some embodiments, can contain one or more asymmetric centers and thus give rise to enantiomers, diastereomers, and other stereoisomeric forms that are defined, in terms of absolute stereochemistry, as (R)- or(S)—. In certain embodiments, the conjugate is of R-configuration. In certain embodiments, the conjugate is of S-configuration. Unless stated otherwise, it is intended that all stereoisomeric forms of the conjugates are contemplated. When the conjugates contain alkene double bonds, and unless specified otherwise, it is intended that both E and Z geometric isomers (e.g., cis or trans) and/or optical isomers are included. In certain embodiments, for example, D and A of a conjugate are arranged in a relative cis orientation. In certain embodiments, D and A of a conjugate are arranged in a relative trans orientation. Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

Further, in each of the foregoing and following embodiments, it is to be understood that the formulae include and represent not only all pharmaceutically acceptable salts of the conjugates, but also include any and all hydrates and/or solvates of the conjugate formulae or salts thereof. Indeed, hydrates, solvates, and N-oxides of the conjugates are also contemplated. The term “solvate” means a conjugate, or a salt thereof, that further includes a stoichiometric or non-stoichiometric amount of solvent bound by non-covalent intermolecular forces. Where the solvent is water, the solvate is a hydrate.

It will be appreciated that certain functional groups, such as the hydroxy, amino, and like groups, form complexes and/or coordination compounds with water and/or various solvents, in the various physical forms of the conjugates. Accordingly, the above formulae are to be understood to include and represent those various hydrates and/or solvates.

In each of the foregoing and following embodiments, it is also to be understood that the formulae include and represent any and all crystalline forms, partially crystalline forms, and non-crystalline and/or amorphous forms of the conjugates.

### Compositions

Further provided is a composition. The composition comprises an above-described (1) mFc fragment, and/or conjugate (e.g., a conjugate of Formula I) or the pharmaceutically acceptable salts thereof, and (2) one or more pharmaceutically acceptable excipients or carriers. The term “composition” generally refers to any product comprising more than one ingredient, including the conjugate. The compositions can be prepared from isolated mFc fragments, conjugates, or from salts, solutions, hydrates, solvates, and other forms of the conjugates.

Compositions contain an effective amount of mFc fragment(s) and/or conjugate(s) hereof as described herein and optionally one or more other therapeutic agents included in a pharmaceutically acceptable carrier. The term “pharmaceutically acceptable carrier” means one or more compatible solid or liquid fillers, diluents or encapsulating substances which are suitable for administration to a subject. The term “carrier” denotes an organic or inorganic ingredient, natural or synthetic, with which the active ingredient is combined to facilitate the application. The components of the compositions also can be commingled with the mFc fragment(s)/conjugate(s) hereof, and with each other, in a manner such that there is no interaction which would substantially impair the desired pharmaceutical efficiency.

In certain embodiments, the composition comprises a plurality of conjugates (e.g., two or more) and a pharmaceutically acceptable excipient. In certain embodiments, a composition further comprises at least one additional pharmaceutically active agent. The at least one additional pharmaceutically active agent can be an agent useful in the treatment of viral infection, for example.

Compositions can be prepared by combining one or more conjugates with a pharmaceutically acceptable excipient and, optionally, one or more additional pharmaceutically active agents in accordance with methods known in the art and described herein below.

The compositions hereof can comprise one or more pharmaceutically acceptable carriers, adjuvants, diluents, excipients, and/or vehicles (e.g., conventional nontoxic pharmaceutically acceptable carriers, adjuvants, and vehicles), and combinations thereof. Any pharmaceutically acceptable carriers, diluents, and excipients as known in the art can be used. Examples include, but are not limited to, an excipient, a color additive, a preservative, and a stabilizer. More specific examples include crystal cellulose, calcium carmellose, sodium carmellose, hydropropylcellulose, hydroxypropylmethylcellulose, ethylcellulose, and magnesium stearate.

Solutions of the mFc fragment, conjugate, or composition can be aqueous, optionally mixed with a nontoxic surfactant, and/or can contain carriers or excipients, such as salts, carbohydrates and buffering agents (preferably at a pH of from 3 to 9), but, for some applications, they can be more suitably formulated as a sterile non-aqueous solution or as a dried form to be used in conjunction with a suitable vehicle, such as sterile, pyrogen-free water, or phosphate-buffered saline. For example, dispersions can be prepared in glycerol, liquid PEGs, triacetin, and mixtures thereof and in oils. Under ordinary conditions of storage and use, these preparations can further contain a preservative to prevent the growth of microorganisms.

The mFc fragments and conjugates can each be formulated as compositions and administered to a mammalian host, such as a human patient, in a variety of forms adapted to the chosen route of administration. The compositions can be formulated, e.g., for a given route of administration, and manufactured in accordance with methods in the art and described, for example, in Remington, The Science and Practice of Pharmacy, 22^{nd} edition (2012). The composition can be an infusion or an injectable composition, such as a composition that can be injected subcutaneously or intravenously.

In certain embodiments, the composition is formulated to be administered subcutaneously. In certain embodiments, the composition is formulated to be administered orally. In certain embodiments, the composition is formulated to be administered intramuscularly, intravenously, intraarterially, intranasally, intraperitoneally, or as any other art-recognized route of parenteral administration.

The composition can be systemically administered in combination with a pharmaceutically acceptable vehicle. The percentages of the components of the compositions and preparations can vary and can be between about 1 to about 99% weight of the active ingredient(s) (e.g., the compound or conjugate) and a binder, an excipient, a disintegrating agent, a lubricant, and/or a sweetening agent (as are known in the art). The amount of active mFc fragment and/or conjugate in such therapeutically useful compositions is such that an effective dosage level can be obtained (e.g., in the serum or targeted tissue or cell).

Illustrative means of parenteral administration include needle (including microneedle) injectors, needle-free injectors and infusion techniques, as well as any other means of parenteral administration recognized in the art. Parenteral formulations are typically aqueous solutions, which can contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, can readily be accomplished using standard pharmaceutical techniques well-known to those skilled in the art.

The pharmaceutical dosage forms suitable for administration can include sterile aqueous solutions or dispersions or sterile powders comprising the active ingredients that are adapted for the extemporaneous preparation of sterile injectable or infusible solutions or dispersions, optionally encapsulated in liposomes, nanocrystals, or polymeric nanoparticles. In all cases, the ultimate dosage form should be sterile, fluid, and stable under the conditions of manufacture and storage. The liquid carrier or vehicle can be a solvent or liquid dispersion medium comprising, for example and without limitation, water, electrolytes, sugars, ethanol, a polyol (e.g., glycerol, propylene glycol, liquid polyethylene glycols, and the like), vegetable oils, nontoxic glyceryl esters, and/or suitable mixtures thereof. In at least one embodiment, the desired fluidity can be maintained by the formation of liposomes, by the maintenance of the required particle size in the case of dispersions or by the use of surfactants.

Sterile injectable solutions can be prepared by incorporating the compositions in the required amount of the appropriate solvent with one or more of the other ingredients set forth above, as required, followed by filter sterilization. In the case of sterile powders for the preparation of sterile injectable solutions, vacuum-drying and freeze-drying techniques can be employed, which can yield a powder of the active ingredient plus any additional desired ingredient present in the previously sterile-filtered solutions.

### Methods

Still further provided is a method of delivering an immune effector to a virus or a cell infected with virus in a subject. The method can comprise administering, such as orally or intravenously, to the subject: (1) an effective amount of the above-described mFc fragment, the above-described conjugate or pharmaceutically acceptable salt thereof, or an above-described composition; and (2) a pharmaceutically acceptable carrier. The immune effector can be any of the mFc fragments hereof.

The subject can be an animal, such as a mammal, e.g., a human. The subject can be in need of an immune effector. The subject can be infected with a virus. The virus can be influenza. The virus can be Influenza A. The virus can be Influenza B.

The method of delivering an immune effector to a virus or a cell infected with a virus can be used to inhibit the full development of a viral infection (e.g., prophylactically). Signs and/or symptoms of a viral infection in the subject can be ameliorated (e.g., by reducing the number and/or severity of signs and/or symptoms) by administration of an effective amount of the mFc fragments, conjugates (or pharmaceutically acceptable salts thereof), and/or compositions hereof.

Methods for treating a viral infection in a subject (or treating a subject having a viral infection or cell infected with a virus) are also provided. Such methods can comprise administering, such as orally or intravenously, to the subject: (1) an effective amount of the above-described mFc fragment, the above-described conjugate or pharmaceutically acceptable salt thereof, or an above-described composition; and (2) a pharmaceutically acceptable carrier.

The subject can be an animal, such as a mammal, e.g., a human. The subject can be in need of an immune effector. The subject can be infected with a virus (e.g., an Influenza virus). The viral infection can be an influenza infection. The viral infection can be an Influenza A infection. The viral infection can be an Influenza B infection.

The terms “treat,” “treating,” “treated,” or “treatment” (with respect to a disease or condition) is an approach for obtaining beneficial or desired results including and preferably clinical results and includes, but is not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival and/or prophylactic or preventative treatment.

An “effective amount” refers to any amount that is sufficient to achieve a desired biological effect. Combined with the teachings provided herein, by choosing among the various mFc fragments and conjugates and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the infection being treated, the particular mFc fragment, conjugate, or composition being administered, concurrently or sequential treatments being administered, the size of the subject, and/or the severity of the infection or condition. One of ordinary skill in the art can empirically determine the effective amount of a particular mFc fragment, conjugate, and/or other therapeutic agent without necessitating undue experimentation. A maximum dose can be used, that is, the highest safe dose according to some medical judgment. Multiple doses per day can be used to achieve appropriate systemic levels of mFc fragment and/or conjugate. Appropriate systemic levels can be determined by, for example, measurement of the patient's peak or sustained plasma level of the drug. “Dose” and “dosage” are used interchangeably herein.

Generally, daily oral doses of a mFc fragment and/or conjugate are, for human subjects, from about 0.01 milligrams/kg per day to 1,000 milligrams/kg per day. Oral doses in the range of 0.5 to 50 milligrams/kg, in one or more administrations per day, can yield therapeutic results. Dosage can be adjusted appropriately to achieve desired drug levels, local or systemic, depending upon the mode of administration. For example, intravenous administration can vary from one order to several orders of magnitude lower dose per day. If the response in a subject is insufficient at such doses, even higher doses (or effective higher doses by a different, more localized delivery route) can be employed to the extent that patient tolerance permits. Multiple doses per day are contemplated to achieve appropriate systemic levels of the mFc fragment and/or conjugate, such as twice daily doses.

An “effective amount” (or “effective amount to treat (therapeutically)”) of a mFc fragment and/or conjugate (used interchangeably herein with “compound”) with respect to use in treatment, refers to an amount of the compound in a preparation which, when administered as part of a desired dosage regimen (to a mammal, such as a human) alleviates a symptom, ameliorates a condition, or slows the onset of infection conditions according to clinically acceptable standards for the disorder or condition to be treated, e.g., at a reasonable benefit/risk ratio applicable to any medical treatment.

For any mFc fragment and/or conjugate, an (e.g., therapeutically) effective amount can be initially determined from animal models. An effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

For clinical use, any compound can be administered in an amount equal or equivalent to 0.2-2,000 milligram (mg) of compound per kilogram (kg) of body weight of the subject per day. The compounds can be administered in a dose equal or equivalent to 2-2,000 mg of compound per kg body weight of the subject per day. The compounds can be administered in a dose equal or equivalent to 20-2,000 mg of compound per kg body weight of the subject per day. The compounds can be administered in a dose equal or equivalent to 50-2,000 mg of compound per kg body weight of the subject per day. The compounds can be administered in a dose equal or equivalent to 100-2,000 mg of compound per kg body weight of the subject per day. The compounds can be administered in a dose equal or equivalent to 200-2,000 mg of compound per kg body weight of the subject per day. In certain embodiments, dosages can range from about 0.01 to about 20 mg of compound per kg body weight of the subject, such as from about 0.1 to about 10 of compound per kg body weight of the subject.

Where a precursor or prodrug of a compound is to be administered, it is administered in an amount that is equivalent to, i.e., sufficient to deliver, the above-stated amounts of the compound.

The formulations of the compounds can be administered to human subjects in therapeutically effective amounts. Typical dose ranges are from about 0.01 microgram/kg to about 20 mg/kg of body weight per day. The dosage of drug to be administered is likely to depend on such variables as the type and extent of the disorder, the overall health status of the particular subject, the specific compound being administered, the excipients used to formulate the compound, and its route of administration. Routine experiments can be used to optimize the dose and dosing frequency for any particular compound.

The compound can be administered at a concentration in the range from about 0.001 microgram/kg to greater than about 500 mg/kg. For example, the concentration can be 0.001 microgram/kg, 0.01 microgram/kg, 0.05 microgram/kg, 0.1 microgram/kg, 0.5 microgram/kg, 1.0 microgram/kg, 10.0 microgram/kg, 50.0 microgram/kg, 100.0 microgram/kg, 500 microgram/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 60.0 mg/kg, 70.0 mg/kg, 80.0 mg/kg, 90.0 mg/kg, 100.0 mg/kg, 150.0 mg/kg, 200.0 mg/kg, 250.0 mg/kg, 300.0 mg/kg, 350.0 mg/kg, 400.0 mg/kg, 450.0 mg/kg, to greater than about 500.0 mg/kg or any incremental value thereof. It is to be understood that all values and ranges between these values and ranges are meant to be encompassed.

The compound can be administered at a dosage in the range from about 0.2 milligram/kg/day to greater than about 100 mg/kg/day. For example, the dosage can be 0.2 mg/kg/day to 100 mg/kg/day, 0.2 mg/kg/day to 50 mg/kg/day, 0.2 mg/kg/day to 25 mg/kg/day, 0.2 mg/kg/day to 10 mg/kg/day, 0.2 mg/kg/day to 7.5 mg/kg/day, 0.2 mg/kg/day to 5 mg/kg/day, 0.25 mg/kg/day to 100 mg/kg/day, 0.25 mg/kg/day to 50 mg/kg/day, 0.25 mg/kg/day to 25 mg/kg/day, 0.25 mg/kg/day to 10 mg/kg/day, 0.25 mg/kg/day to 7.5 mg/kg/day, 0.25 mg/kg/day to 5 mg/kg/day, 0.5 mg/kg/day to 50 mg/kg/day, 0.5 mg/kg/day to 25 mg/kg/day, 0.5 mg/kg/day to 20 mg/kg/day, 0.5 mg/kg/day to 15 mg/kg/day, 0.5 mg/kg/day to 10 mg/kg/day, 0.5 mg/kg/day to 7.5 mg/kg/day, 0.5 mg/kg/day to 5 mg/kg/day, 0.75 mg/kg/day to 50 mg/kg/day, 0.75 mg/kg/day to 25 mg/kg/day, 0.75 mg/kg/day to 20 mg/kg/day, 0.75 mg/kg/day to 15 mg/kg/day, 0.75 mg/kg/day to 10 mg/kg/day, 0.75 mg/kg/day to 7.5 mg/kg/day, 0.75 mg/kg/day to 5 mg/kg/day, 1.0 mg/kg/day to 50 mg/kg/day, 1.0 mg/kg/day to 25 mg/kg/day, 1.0 mg/kg/day to 20 mg/kg/day, 1.0 mg/kg/day to 15 mg/kg/day, 1.0 mg/kg/day to 10 mg/kg/day, 1.0 mg/kg/day to 7.5 mg/kg/day, 1.0 mg/kg/day to 5 mg/kg/day, 2 mg/kg/day to 50 mg/kg/day, 2 mg/kg/day to 25 mg/kg/day, 2 mg/kg/day to 20 mg/kg/day, 2 mg/kg/day to 15 mg/kg/day, 2 mg/kg/day to 10 mg/kg/day, 2 mg/kg/day to 7.5 mg/kg/day, or 2 mg/kg/day to 5 mg/kg/day.

The compound can be administered at a dosage in the range from about 0.25 milligram/kg/day to about 25 mg/kg/day. For example, the dosage can be 0.25 mg/kg/day, 0.5 mg/kg/day, 0.75 mg/kg/day, 1.0 mg/kg/day, 1.25 mg/kg/day, 1.5 mg/kg/day, 1.75 mg/kg/day, 2.0 mg/kg/day, 2.25 mg/kg/day, 2.5 mg/kg/day, 2.75 mg/kg/day, 3.0 mg/kg/day, 3.25 mg/kg/day, 3.5 mg/kg/day, 3.75 mg/kg/day, 4.0 mg/kg/day, 4.25 mg/kg/day, 4.5 mg/kg/day, 4.75 mg/kg/day, 5 mg/kg/day, 5.5 mg/kg/day, 6.0 mg/kg/day, 6.5 mg/kg/day, 7.0 mg/kg/day, 7.5 mg/kg/day, 8.0 mg/kg/day, 8.5 mg/kg/day, 9.0 mg/kg/day, 9.5 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, 30 mg/kg/day, 31 mg/kg/day, 32 mg/kg/day, 33 mg/kg/day, 34 mg/kg/day, 35 mg/kg/day, 36 mg/kg/day, 37 mg/kg/day, 38 mg/kg/day, 39 mg/kg/day, 40 mg/kg/day, 41 mg/kg/day, 42 mg/kg/day, 43 mg/kg/day, 44 mg/kg/day, 45 mg/kg/day, 46 mg/kg/day, 47 mg/kg/day, 48 mg/kg/day, 49 mg/kg/day, or 50 mg/kg/day.

The compound can be administered in concentrations that range from 0.01 micromolar to greater than or equal to 500 micromolar. For example, the dose can be 0.01 micromolar, 0.02 micromolar, 0.05 micromolar, 0.1 micromolar, 0.15 micromolar, 0.2 micromolar, 0.5 micromolar, 0.7 micromolar, 1.0 micromolar, 3.0 micromolar, 5.0 micromolar, 7.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 60.0 micromolar, 70.0 micromolar, 80.0 micromolar, 90.0 micromolar, 100.0 micromolar, 150.0 micromolar, 200.0 micromolar, 250.0 micromolar, 300.0 micromolar, 350.0 micromolar, 400.0 micromolar, 450.0 micromolar, to greater than about 500.0 micromolar or any incremental value thereof. It is to be understood that all values and ranges between these values and ranges are meant to be encompassed.

The compound can be administered at concentrations that range from 0.10 microgram/mL to 500.0 microgram/mL. For example, the concentration can be 0.10 microgram/mL, 0.50 microgram/mL, 1 microgram/mL, 2.0 microgram/mL, 5.0 microgram/mL, 10.0 microgram/mL, 20 microgram/mL, 25 microgram/mL. 30 microgram/mL, 35 microgram/mL, 40 microgram/mL, 45 microgram/mL, 50 microgram/mL, 60.0 microgram/mL, 70.0 microgram/mL, 80.0 microgram/mL, 90.0 microgram/mL, 100.0 microgram/mL, 150.0 microgram/mL, 200.0 microgram/mL, 250.0 g/mL, 250.0 micro gram/mL, 300.0 microgram/mL, 350.0 microgram/mL, 400.0 microgram/mL, 450.0 microgram/mL, to greater than about 500.0 microgram/mL or any incremental value thereof. It is to be understood that all values and ranges between these values and ranges are meant to be encompassed.

The formulations can be administered in pharmaceutically acceptable solutions, which can routinely contain pharmaceutically acceptable concentrations of salt, buffering agents, preservatives, compatible carriers, adjuvants, and optionally other therapeutic ingredients. For use in therapy, an effective amount of the conjugate can be administered to a subject by any mode that delivers the conjugate to the desired surface. Administering a composition can be accomplished by any means known to the skilled artisan. Routes of administration include, but are not limited to, intravenous, intramuscular, intraperitoneal, intravesical (urinary bladder), oral, subcutaneous, direct injection (for example, into a tumor or abscess), mucosal (e.g., topical to eye), inhalation, and topical.

For oral administration, the compounds can be formulated readily by combining the mFc fragment and/or conjugate(s) with pharmaceutically acceptable excipients well-known in the art. Such excipients enable the compound to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents can be added, such as the cross-linked PVP, agar, or alginic acid or a salt thereof such as sodium alginate. Optionally the oral formulations can also be formulated in saline or buffers, e.g., EDTA for neutralizing internal acid conditions, or can be administered without any excipients.

Also contemplated are oral dosage forms of the compounds. The compound can be chemically modified so that oral delivery of the derivative is efficacious. Generally, the chemical modification contemplated is the attachment of at least one moiety to the compound itself, where said moiety permits (a) inhibition of acid hydrolysis; and (b) uptake into the blood stream from the stomach or intestine. Also desired is the increase in overall stability of the compound and increased in circulation time in the body. Examples of such moieties include PEG, copolymers of ethylene glycol and propylene glycol, carboxymethyl cellulose, dextran, polyvinyl alcohol, PVP and polyproline. Abuchowski and Davis, “Soluble Polymer-Enzyme Adducts,” In: Enzymes as Drugs, Hocenberg and Roberts, eds., Wiley-Interscience, New York, N.Y., pp. 367-383 (1981); Newmark et al., Preparation and properties of adducts of streptokinase and streptokinase-plasmin complex with polyethylene glycol and pluronic polyol F38, J Appl Biochem 4:185-189 (1982). Other polymers that could be used are poly-1,3-dioxolane and poly-1,3,6-tioxocane. For pharmaceutical usage, as indicated above, PEG moieties are suitable.

The location of release of a compound hereof can be the stomach, the small intestine (e.g., the duodenum, the jejunum, or the ileum), or the large intestine. One skilled in the art has available formulations, which will not dissolve in the stomach, yet will release the material in the duodenum or elsewhere in the intestine. The release can avoid the deleterious effects of the stomach environment, either by protection of the compound or by release of the compound beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings can be used as mixed films.

A coating or mixture of coatings can also be used on tablets, which are not intended for protection against the stomach. This can include sugar coatings, or coatings which make the tablet easier to swallow. Capsules can consist of a hard shell (such as gelatin) for delivery of dry therapeutic (e.g., powder); for liquid forms, a soft gelatin shell can be used. The shell material of cachets could be thick starch or other edible paper. For pills, lozenges, molded tablets or tablet triturates, moist massing techniques can be used.

The compound can be included in the formulation as fine multi-particulates in the form of granules or pellets of particle size about 1 mm. The formulation of the material for capsule administration could also be as a powder, lightly compressed plugs or even as tablets. Therapeutic agent could be prepared by compression.

Colorants and flavoring agents may all be included. For example, the compound can be formulated (such as by liposome or microsphere encapsulation) and then further contained within an edible product, such as a refrigerated beverage containing colorants and flavoring agents.

One may dilute or increase the volume of the compound with an inert material. These diluents can include carbohydrates, especially mannitol, a-lactose, anhydrous lactose, cellulose, sucrose, modified dextrans and starch. Certain inorganic salts also can be used as fillers including calcium triphosphate, magnesium carbonate and sodium chloride. Some commercially available diluents are Fast-Flo, Emdex, STA-Rx 1500, Emcompress and Avicell.

Disintegrants can be included in the formulation of therapeutic agent into a solid dosage form. Materials used as disintegrates include, but are not limited to, starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrant is the insoluble cationic exchange resin. Powdered gums can be used as disintegrants and as binders and these can include powdered gums such as agar, Karaya or tragacanth. Alginic acid and its sodium salt are also useful as disintegrants.

Binders can be used to hold the compound together to form a hard tablet and include materials from natural products such as acacia, tragacanth, starch and gelatin. Others include methyl cellulose (MC), ethyl cellulose (EC) and carboxymethyl cellulose (CMC). PVP and HPMC can both be used in alcoholic solutions to granulate therapeutic agent.

An anti-frictional agent can be included in the formulation of therapeutic to prevent sticking during the formulation process. Lubricants can be used as a layer between therapeutic agent and the die wall, and these can include, but are not limited to, stearic acid, including its magnesium and calcium salts, polytetrafluoroethylene (PTFE), liquid paraffin, vegetable oils and waxes. Soluble lubricants can also be used, such as sodium lauryl sulfate, magnesium lauryl sulfate, polyethylene glycol of various molecular weights, Carbowax 4000 and 6000.

Glidants, which can improve the flow properties of the drug during formulation and aid rearrangement during compression, can be added. The glidants can include starch, talc, pyrogenic silica and hydrated silicoaluminate.

To aid dissolution of therapeutic agent into the aqueous environment a surfactant can be added as a wetting agent. Surfactants can include anionic detergents, such as sodium lauryl sulfate, dioctyl sodium sulfosuccinate and dioctyl sodium sulfonate. Cationic detergents which can be used include benzalkonium chloride and benzethonium chloride. Potential non-ionic detergents that can be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound or derivative thereof either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Microspheres formulated for oral administration can also be used. Such microspheres have been well defined in the art. All formulations for oral administration should be in dosages suitable for such administration.

For buccal administration, the compositions can take the form of tablets or lozenges formulated in conventional manner.

For administration by inhalation, compounds can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

Also contemplated is pulmonary delivery of the compounds (or salts thereof). The compound is delivered to the lungs of a mammal while inhaling and traverses across the lung epithelial lining to the blood stream. Other reports of inhaled molecules include Adjei & Garren, Pulmonary delivery of peptide drugs: Effect of particle size on bioavailability of leuprolide acetate in healthy male volunteers, J Pharmaceutical Research 7:565-569 (1990); Adjei et al., Bioavailability of leuprolide following intratracheal administration to beagle dogs, International J Pharmaceutics 63:135-144 (1990) (leuprolide acetate); Braquet et al., Effect of endothelin-1 on blood pressure and bronchopulmonary system of the guinea pig, J Cardiovascular Pharmacology 13 (suppl. 5): 143-146 (1989) (endothelin-1); Hubbard et al., Annals of Internal Medicine 3:206-212 (1989) (al-antitrypsin); Smith et al., Pulmonary deposition and clearance of aerosolized alpha-1-proteinase inhibitor administered to dogs and to sheep, J Clinical Investigation 84:1145-1146 (1989) (a-1-proteinase); Oswein et al., Aerosolization of Proteins, Proceedings of Symposium on Respiratory Drug Delivery II, Keystone, Colorado, March 1990 (recombinant hepatocyte growth hormone); Debs et al., Lung-specific delivery of cytokines induces sustained pulmonary and systemic immunomodulation in rats, J Immunology 140:3482-3488 (1988) (interferon-gamma and tumor necrosis factor alpha) and U.S. Pat. No. 5,284,656 (granulocyte colony stimulating factor; incorporated herein by reference). A method and composition for pulmonary delivery of drugs for systemic effect is described in U.S. Pat. No. 5,451,569 (specifically incorporated herein by reference for its disclosure regarding same).

Contemplated for use are a wide range of mechanical devices designed for pulmonary delivery of therapeutic products, including but not limited to nebulizers, metered dose inhalers, and powder inhalers, all of which are familiar to those skilled in the art.

Nasal delivery of a composition is also contemplated. Nasal delivery allows the passage of a composition to the blood stream directly after administering therapeutic product to the nose, without the necessity for deposition of the compound in the lung. Formulations for nasal delivery include those with dextran or cyclodextran.

The compound, when systemic delivery is desirable, can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the compound can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethylcellulose, sorbitol, or dextran. Optionally, the suspension can also contain suitable stabilizers or agents which increase the solubility of the compounds to allow for the preparation of highly concentrated solutions.

Alternatively, the compound can be in powder form for constitution with a suitable vehicle, e.g., sterile pyrogen-free water, before use.

The compound can also be formulated in rectal or vaginal compositions such as suppositories or retention enemas, e.g., containing conventional suppository bases such as cocoa butter or other glycerides.

In addition to the formulations described above, a compound can also be formulated as a depot preparation. Such long-acting formulations can be formulated with suitable polymeric or hydrophobic materials (for example as an emulsion in an acceptable oil) or ion exchange resins, or as sparingly soluble derivatives, for example, as a sparingly soluble salt.

The compositions also can comprise suitable solid or gel phase or excipients. Examples of such excipients include, but are not limited to, calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro) capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The compositions are suitable for use in a variety of drug delivery systems. For a brief review of methods for drug delivery, see Langer, New methods of drug delivery, Science 249 (4976): 1527-1533 (1990).

The compound and optionally one or more other therapeutic agents can be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Therapeutic agent(s), including specifically, but not limited to, the compound, can be provided in particles. “Particles” as used herein means nanoparticles or microparticles (or in some instances larger particles) that can consist in whole or in part of the compound or the other therapeutic agent(s) as described herein. The particles can contain therapeutic agent(s) in a core surrounded by a coating, including, but not limited to, an enteric coating. Therapeutic agent(s) also can be dispersed throughout the particles. Therapeutic agent(s) also can be adsorbed into the particles. The particles can be of any order release kinetics, including zero-order release, first-order release, second-order release, delayed release, sustained release, immediate release, and any combination thereof, etc. The particle can include, in addition to therapeutic agent(s), any of those materials routinely used in the art of pharmacy and medicine, including, but not limited to, erodible, nonerodible, biodegradable, or nonbiodegradable material or combinations thereof. The particles can be microcapsules which contain the compound in a solution or in a semi-solid state. The particles can be of virtually any shape.

Both non-biodegradable and biodegradable polymeric materials can be used in the manufacture of particles for delivering therapeutic agent(s). Such polymers can be natural or synthetic polymers. The polymer is selected based on the period of time over which release is desired. Bioadhesive polymers of particular interest include bioerodible hydrogels described in Sawhney et al., Bioerodible hydrogels based on photopolymerized poly (ethylene glycol)-co-poly (.alpha.-hydroxy acid) diacrylate macromers, Macromolecules 26 (4): 581-587 (1993), the teachings of which are specifically incorporated by reference herein. These include polyhyaluronic acids, casein, gelatin, glutin, polyanhydrases, polyacrylic acid, alginate, chitosan, poly (methyl methacrylates), poly (ethyl methacrylates), poly (butylmethacrylate), poly(isobutyl methacrylate), poly (hexylmethacrylate), poly (isodecyl methacrylate), poly (lauryl methacrylate), poly (phenyl methacrylate), poly(methyl acrylate), poly(isopropyl acrylate), poly(isobutyl acrylate), and poly (octadecyl acrylate).

Compound(s) can be contained in controlled-release systems. The term “controlled release” is intended to refer to any drug-containing formulation in which the manner and profile of drug release from the formulation are controlled. This refers to immediate as well as non-immediate release formulations, with non-immediate release formulations including, but not limited to, sustained release and delayed release formulations. The term “sustained release” (also referred to as “extended release”) is used in its conventional sense to refer to a drug formulation that provides for gradual release of a drug over an extended period of time, and that can result in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

Use of a long-term sustained release implant can be particularly suitable for treatment of chronic conditions. “Long-term” release, as used herein, means that the implant is constructed and arranged to deliver therapeutic levels of the active ingredient for at least 7 days, and up to 30-60 days. Long-term sustained release implants are well-known to those of ordinary skill in the art and include some of the release systems described above.

### Certain Definitions

As used herein, the following terms and phrases shall have the meanings set forth below. Unless defined otherwise, all technical and scientific terms used herein have the same meaning as commonly understood to one of ordinary skill in the art.

The term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value) provided that one of ordinary skill in the art would consider equivalent to the recited value (e.g., having the same function or result). The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. Thus, for example, references to “the method” includes one or more methods and/or steps of the type, which are described herein and/or which will become apparent to those ordinarily skilled in the art upon reading the disclosure.

The term “or” is used to refer to a nonexclusive “or” unless otherwise indicated. In addition, it is to be understood that the phraseology or terminology employed herein, and not otherwise defined, is for the purpose of description only and not of limitation.

An “alkyl group” is a saturated, partially saturated, or unsaturated straight chain or branched non-cyclic hydrocarbon having from 1 to 10 carbon atoms (C₁-C₁₀ alkyl), from 1 to 8 carbons (C₁-C₈alkyl), from 1 to 6 (C₁-C₆ alkyl), 1 to 4 (C₁-C₄ alkyl), 1 to 3 (C₁-C₃ alkyl), or 2 to 6 (C₂-C₆ alkyl) carbon atoms. In some embodiments, the alkyl group has monovalency. Examples of alkyl groups with monovalency include —CH₃, —CH₂CH₃, and the like. Monovalent alkyls may be found on substitutions in the chain of linker, L, for example. In some embodiments, the alkyl group has bivalency, such as when found in the chain of the linker, L. Examples of alkyl groups with bivalency include, but are not limited to, —CH₂—, —CH₂CH₂—, and the like. In some embodiments, the alkyl group is a saturated alkyl group. In some embodiments, an alkyl group is an unsaturated alkyl group, also termed an alkenyl group or an alkynyl group.

The terms “patient” and “subject” are used interchangeably and include a human patient, a laboratory animal, such as a rodent (e.g., mouse, rat, or hamster), a rabbit, a monkey, a chimpanzee, a domestic animal, such as a dog, a cat, or a rabbit, an agricultural animal, such as a cow, a horse, a pig, a sheep, or a goat, or a wild animal in captivity, such as a bear, a panda, a lion, a tiger, a leopard, an elephant, a zebra, a giraffe, a gorilla, a dolphin, or a whale. The patient to be treated is preferably a mammal, in particular a human being.

The terms “protein,” “polypeptide” and “peptide” refer to compounds comprising amino acids joined via peptide bonds and are used interchangeably.

Any use of section headings and subheadings is solely for ease of reference and is not intended to limit any disclosure made in one section to that section only; rather, any disclosure made under one section heading or subheading is intended to constitute a disclosure under each and every other section heading or subheading.

Various modifications and variations of the described compounds, compositions, methods, and uses of the technology will be apparent to those skilled in the art without departing from the scope and spirit of the technology as described. Although the technology has been described in connection with specific exemplary embodiments, the invention as claimed should not be unduly limited to such specific embodiments. Indeed, various modifications of the described modes for carrying out the invention that are obvious to those skilled in the art are intended to be within the scope of the following claims.

The terms and expressions, which have been employed, are used as terms of description and not of limitation. In this regard, where certain terms are defined and are described or discussed elsewhere, the definitions and all descriptions and discussions are intended to be attributed to such terms. There also is no intention in the use of such terms and expressions of excluding any equivalents of the features shown and described or portions thereof.

Further, all publications and patents mentioned herein are incorporated by reference in their entireties for all purposes. In the event of inconsistent usages between this document and those documents so incorporated by reference, the usage in the incorporated reference should be considered supplementary to that of this document; for irreconcilable inconsistencies, the usage in this document controls.

TABLE 2
List of abbreviations

| | |
|---|---|
| MeOH | Methanol |
| EtOH | Ethanol |
| EtOAc | Ethyl acetate |
| DCM | Dichloromethane |
| ACN | Acetonitrile |
| THF | Tetrahydrofuran |
| TFA | Trifluoroacetic acid |
| TIPS | Triisopropylsilane |
| Py | Pyridine |
| TEA | Triethyl amine |
| DIPEA | N,N-Diisopropylethylamine |
| rt | Room temperature |
| M | Molar |
| DMAP | N,N-Dimethyl aminopyridine |
| PPh₃ | Triphenyl phosphine |
| PEG | Polyethylene glycol |
| ^{t}Bu | tert-Butyl |
| NH₄OAc | Ammonium acetate |
| Equiv. | Equivalence |
| aq. | Aqueous |
| min | Minutes |
| g | Grams |
| mL | Milliliter |
| mmol | Millimoles |
| Qty. | Quantity |
| LC-MS | Liquid chromatography-mass spectrometry |
| ESI | Electron spray ionization |
| m/z | Mass/charge ratio |
| pTSOH | p-Toluene sulfonic acid |
| Na₂CO₃ | Sodium carbonate |
| NaOMe | Sodium methoxide |
| Zan | Zanamivir |
| PBS | Phosphate buffered saline |

### EMBODIMENTS

The following enumerated embodiments, represented by clauses, are non-limiting aspects according to the present disclosure and describe particular embodiments hereof:
Clause A. A monomeric Fc (mFc) fragment of human immunoglobulin G1 (IgG1) which:
(i) has a molecular weight of less than 40 kDa,
(ii) comprises (a) a C_{H}2 domain, (b) a C_{H}2 domain to the C terminus of which a neonatal Fc receptor (FcRn) binding motif from a C_{H}3 domain is attached, (c) a C_{H}3 domain, or (d) both of a C_{H}2 domain and a C_{H}3 domain,
(iii) binds to the FcRn and at least one Fcγ receptor, and
(iv) comprises SEQ ID NO: 1 at an N-terminus of the fragment, optionally substituted with one or two conservative amino acid substitutions.
Clause B. The mFc fragment of clause A, wherein the FcRn binding motif from a C_{H}3 domain is or comprises SEQ ID NO: 2.
Clause C. The mFc fragment of clause A, which comprises at its C-terminus SEQ ID NO: 3, which is optionally substituted with one or two conservative amino acid substitutions.
Clause D. The mFc fragment of any one of clauses 1, 2 or 3, comprising IKAROS family zinc finger 1 (IKS) at amino acid positions corresponding to amino acids 338-340 (KAK) of SEQ ID NO: 4.
Clause E. The mFc fragment of any one of clauses A, B or C, comprising a deletion corresponding to amino acids 341-447 of SEQ ID NO: 4.
Clause F. The mFc fragment of any one of clauses A, B or C, comprising a serine at the amino acid position corresponding to amino acid 431 (C) of SEQ ID NO: 4.
Clause G. The mFc fragment of any one of clauses A, B or C, wherein the at least one Fcγ receptor is FcγRI, FcγRIIa, FcγRIIb, and/or FcγRIII.
Clause H. The mFc fragment of clause A, comprising an amino acid sequence of SEQ ID NO: 5.
Clause I. The mFc fragment of clause A, comprising an amino acid sequence of SEQ ID NO: 6.
Clause J. The mFc fragment of clause A, comprising an amino acid sequence of SEQ ID NO: 7.
Clause K. The mFc fragment of clause A, comprising an amino acid sequence of SEQ ID NO: 8.
Clause L. The mFc fragment of clause A, comprising an amino acid sequence of SEQ ID NO: 9.
Clause M. The mFc fragment of clause A, comprising an amino acid sequence of SEQ ID NO: 10.
Clause N. The mFc fragment of clause A, comprising an amino acid sequence of SEQ ID NO: 11.
Clause O. The mFc fragment of clause A, comprising an amino acid sequence of SEQ ID NO: 12.
Clause P. The mFc fragment of clause A, comprising an amino acid sequence of SEQ ID NO: 13.
Clause Q. The mFc fragment of clause A, comprising an amino acid sequence of SEQ ID NO: 14.
Clause R. The mFc fragment of clause A, comprising an amino acid sequence of SEQ ID NO: 15.
Clause S. The mFc fragment of clause A, comprising at least one mutation that increases affinity to at least one Fcγ receptor.
Clause T. The mFc fragment of clause S, wherein the at least one Fcγ receptor is FcγRIII.
Clause U. The mFc fragment of clause S, wherein the at least one mutation is at least one of S239D, A330L, and I332E.
Clause V. The mFc fragment of clause A, comprising at least one mutation that increases affinity to the FcRn receptor comparative to wild-type.
Clause W. The mFc fragment of clause S or V, wherein the at least one mutation that increases affinity to the FcRn receptor is selected from the group consisting of H433K/N434F/Y436H and H433K/N434F.
Clause X. The mFc fragment of clause A, comprising at least one mutation that increases in vivo stability of the fragment.
Clause Y. The mFc fragment of clause X, wherein the at least one mutation is at least one of L242C, K334C, P343C, and A431C.
Clause Z. The mFc fragment of clause A, which comprises at least one mutation that decreases or eliminates aggregation of the fragment.
Clause AA. The mFc fragment of clause Z, wherein the at least one mutation is at least one of L351S, T366R, L368H, and P395K.
Clause BB. The mFc fragment of clause A, which comprises at least one site for conjugation.
Clause CC. The mFc fragment of clause BB, wherein the at least one site for conjugation is at least one of a cysteine, a serine, a lysine, a tyrosine, and an unnatural amino acid.
Clause DD. A conjugate of formula I:

mFc-L-smL (Formula I)

or a pharmaceutically acceptable salt thereof, wherein:
mFc is a monomeric Fc fragment of any of claims A-CC;
smL is a radical of a small molecule ligand that binds to neuraminidase (NA) or hemagglutinin (HA) on influenza virus or an influenza virus-infected cell; and
L is a linker that is covalently bound to smL and mFc.
Clause EE. The conjugate of clause DD, where smL is a radical of a NA inhibitor or an HA inhibitor.
Clause FF. The conjugate of clause DD or EE, wherein smL is a radical of zanamivir.
Clause GG. The conjugate of clause DD or EE, wherein smL is a radical of oseltamivir, peramivir, or laninamivir.
Clause HH. The conjugate of clause DD, wherein mFc comprises at least one cysteine, lysine, and/or tyrosine via which L is covalently bound to mFc.
Clause II. The conjugate of clause DD, wherein L is covalently bound to mFc via an unnatural amino acid.
Clause JJ. The conjugate of clause DD, wherein L further comprises a spacer.
Clause KK. The conjugate of clause DD, wherein L is non-cleavable.
Clause LL. The conjugate of clause DD, wherein L increases the water-solubility of the conjugate.
Clause MM. The conjugate of clause DD, wherein L comprises one or more of an amino acid, a polyethylene glycol (PEG) monomer, a PEG oligomer, a PEG polymer, or a combination of two or more of the foregoing.
Clause NN. The conjugate of clause DD, wherein L comprises an oligomer of peptidoglycans, glycans, anions, or a combination of two or more of the foregoing.
Clause OO. A composition comprising a conjugate of any one of clauses DD-NN and a pharmaceutically acceptable carrier.
Clause PP. A method of delivering an immune effector to influenza virus or a cell infected with influenza virus in a subject comprising administering to the subject an effective amount of a conjugate of any one of clauses DD-NN or a composition comprising the conjugate and a pharmaceutically acceptable carrier.
Clause QQ. The method of clause PP, wherein the immune effector is the mFc fragment of any one of clauses A-CC.
Clause RR. The method of clause PP, wherein the conjugate or composition is administered intravascularly, orally, or intranasally.
Clause SS. A method of treating a subject having viral infection or a cell infected with a virus comprising administering to the subject an effective amount of a conjugate of any one of clauses 30-40 or a composition comprising the conjugate and a pharmaceutically acceptable carrier.
Clause TT. The method of clause SS, wherein the immune effector is the mFc fragment of any one of clauses A-NN.
Clause UU. The method of clause SS, wherein the conjugate or composition is administered intravascularly, orally, or intranasally.
Clause VV. The method of any one of clauses SS-UU, wherein the conjugate or composition is administered to the subject in a single dose.
Clause WW. The method of any one of clauses SS-UU, wherein the conjugate or composition is administered to the subject in more than one dose.
Clause XX. The method of any one of clauses SS-UU, wherein the virus is an Influenza virus.
Clause YY. The method of any one of clauses SS-UU, wherein the virus is an Influenza A virus.

### EXAMPLES

The following examples serve to illustrate the present disclosure and are not intended to limit the scope of the claimed invention in any way.

### Example 1

Construction and Expression of First-Generation IgG1 Wild-Type and Mutant C_{H}2-C_{H}3 Domains

Construction of IgG1-C_{H}2-C_{H}3-GASDALIE mutant (SEQ ID NO: 5) (Gly236Ala/Ser239Asp/Ala330Leu/Ile332Glu), which is a GASDALIE mutant of SEQ ID NO: 4 (see FIG. 1): This mFc fragment exhibited increased binding affinity to FcγRIIa and FcγRIIIa comparative with wild-type (WT), and subsequently enhanced antibody-dependent cell-mediated cytotoxicity (ADCC) activity.

Construction of IgG1-C_{H}2-C_{H}3-DLE mutant (SEQ ID NO: 6) (Ser239Asp/Ala330Leu/Ile332Glu DLE), which is a DLE mutant of SEQ ID NO: 4 (see FIG. 1): As compared to WT (IgG1-C_{H}2-C_{H}3-WT (SEQ ID NO: 4)), this mFc fragment exhibited increased FcγRIIIa affinity, low binding to inhibitory FcγRIIb, and subsequently enhanced ADCC and antibody-dependent cellular phagocytosis (ADCP) activity.

For the expression studies, all IgG1 C_{H}2-C_{H}3 domain WT and mutants were synthesized and subcloned into a pCMN1.1 vector in operable linkage with a cytomegalovirus (CMV) promoter.

The ligated plasmid DNAs (i.e., SEQ ID NO: 17, which encodes SEQ ID NO: 4, SEQ ID NO: 18, which encodes SEQ ID NO: 5, and SEQ ID NO: 19, which encodes SEQ ID NO; 6) were prepared, and the correct sequences were confirmed prior to transient transfection using CHO—S cells. Five to six days after transfection, cell suspensions were centrifuged at 8,000 rpm for 30 minutes and supernatants were recovered. The IgG1 Fc wild/mutants were purified by protein A affinity chromatography.

Briefly, the culture supernatant of each Fc mutant was passed through 0.22 μm filters before loading onto polypropylene columns packed with Protein A high-capacity agarose resin. The resulting flow-through was collected and passed twice more through the column before any unbound protein was washed away with >10 CV (Column Volume) of 1× PBS. All antibodies were eluted with 3 ml of 100 mM citrate buffer (pH 3.0) and immediately neutralized with 1 ml of 1 M Tris (hydroxymethyl) aminomethane (Tris) (pH 8.0). Samples were buffer-exchanged into 1× PBS using Amicon Ultra-4 (Millipore Sigma, Darmstadt, Germany) spin columns with a 10 kDa cutoff, and the purity of purified samples was assessed by 4-20% gradient sodium dodecyl sulfate-polyacrylamide gel electrophoresis (SDS-PAGE gel). The final Fc proteins were approximately 26 kDa as monomeric form. The excitation coefficient value was 1.4.

### Example 2

### Affinity Measurement of IgG1 Fc WT and Mutants for Fc Gamma Receptors (FcγRs)

Bio Layer Interferometry (BLI) was used to determine equilibrium K_{D} values for Fc WT and mutant binding to FcγRs according to an in-house standard operating procedure known in the art. Binding response, dissociation constant binding K_{D}, and association and dissociation rates of the mAbs were derived from binding kinetics analyzed by BLI using an Octet HTX instrument.

Briefly, HISIK (anti-Penta-HIS)-coated biosensors were loaded with His-tagged human FcγR variants obtained from Beijing Sino Biological Inc. (Beijing, China) (CD16a (176V), CD16a (176F), CD32a (167R), CD32a (167H), and CD32b) in kinetic assay buffer (1× PBS pH7, 0.1% bovine serum albumin (BSA) and 0.05% Tween 20), and the monomeric Fc variants were two-fold serially diluted with the same assay buffer and allowed to bind to FcγR-loaded biosensors. The dissociation phase was then recorded after switching the biosensors to assay buffer. Kinetic data (KD, ka, kd and Rmax) were double-referenced and fit globally to a simple 1:1 Langmuir binding model and determined accordingly, and are shown in Table 3.

TABLE 3
Binding affinities of the Fc fragments with corresponding
Fc gamma receptors determined by Octet assay

| Mono-Fc | | huFcγR | | |
|---|---|---|---|---|
| Variants | SEQ ID NO: | Variants | KD (M) | Full R² |
| Mono-Fc DLE | 6 | CD16a(176F) | 1.81E−08 | 0.9483 |
| | | CD16a(176V) | 9.83E−09 | 0.9541 |
| | | CD32a(167R) | *6.93E−10 | 0.8138 |
| | | CD32a(167H) | *2.11E−10 | 0.8099 |
| | | CD32b | 1.34E−09 | 0.6577 |
| Mono-Fc | 5 | CD16a(176F) | 1.58E−08 | 0.9438 |
| GASDALIE | | CD16a(176V) | 1.62E−08 | 0.9665 |
| | | CD32a(167R) | 2.21E−07 | 0.933 |
| | | CD32a(167H) | *3.61E−06 | 0.7254 |
| | | CD32b | *4.71E−10 | 0.796 |
| Mono-Fc WT | 4 | CD16a(176F) | *<1.0E−12 | 0.652 |
| | | CD16a(176V) | *1.13E−06 | 0.8396 |
| | | CD32a(167R) | *1.34E−08 | 0.8537 |
| | | CD32a(167H) | *5.42E−09 | 0.8529 |
| | | CD32b | *3.76E−06 | |

*Denotes weak binding/inaccurate KD

### Example 3

Conjugation of Fc Fragment with Small Molecule Drug

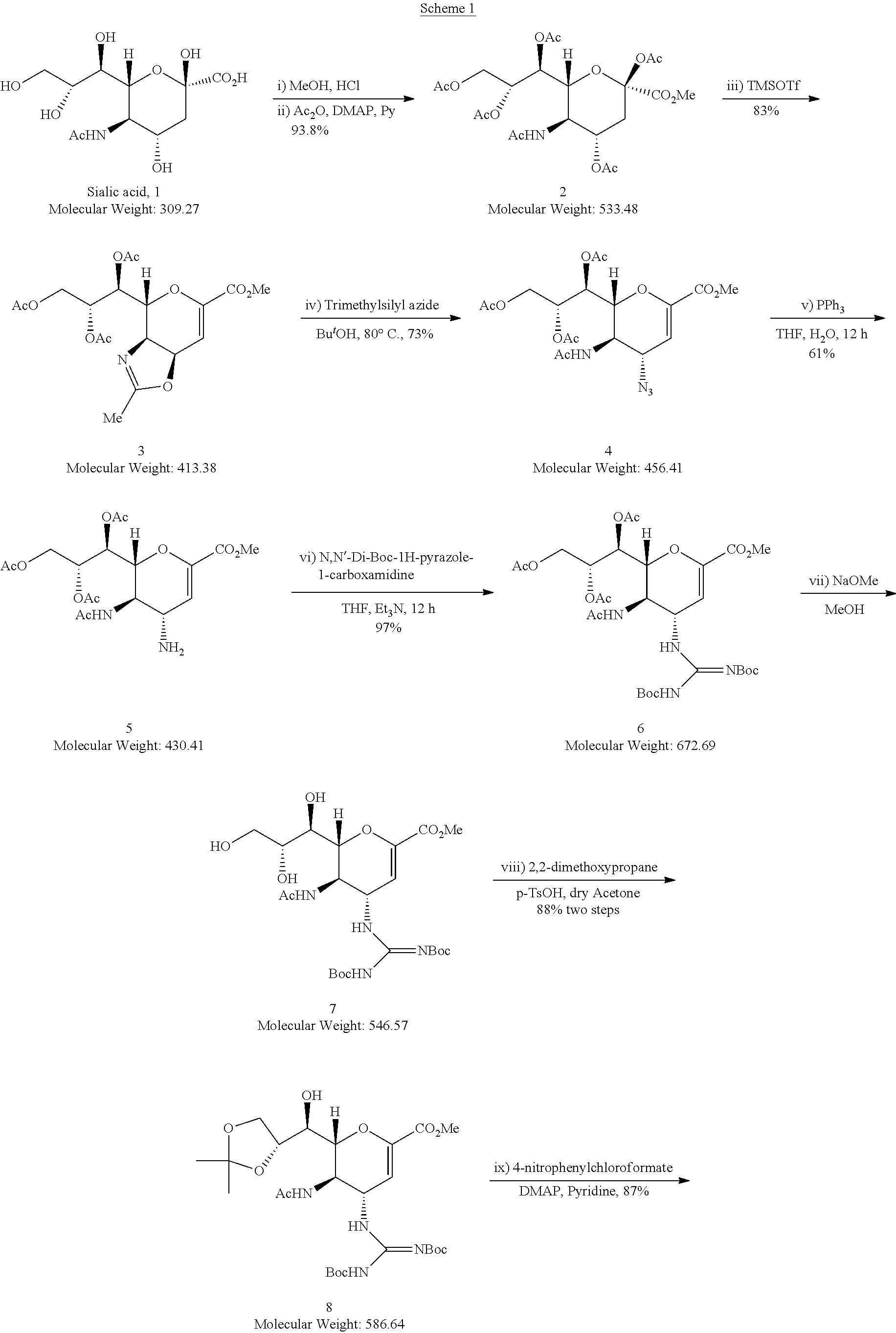

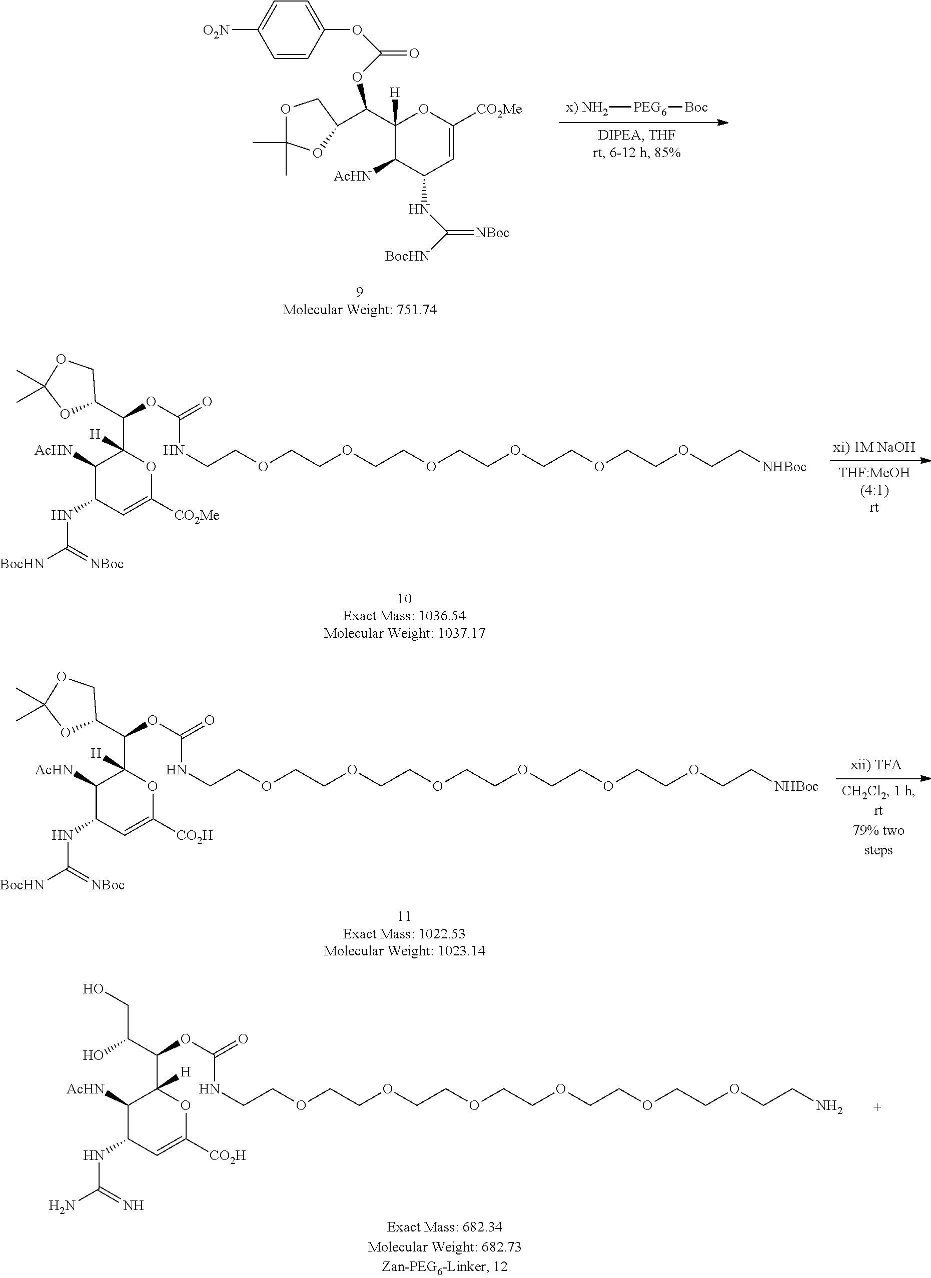

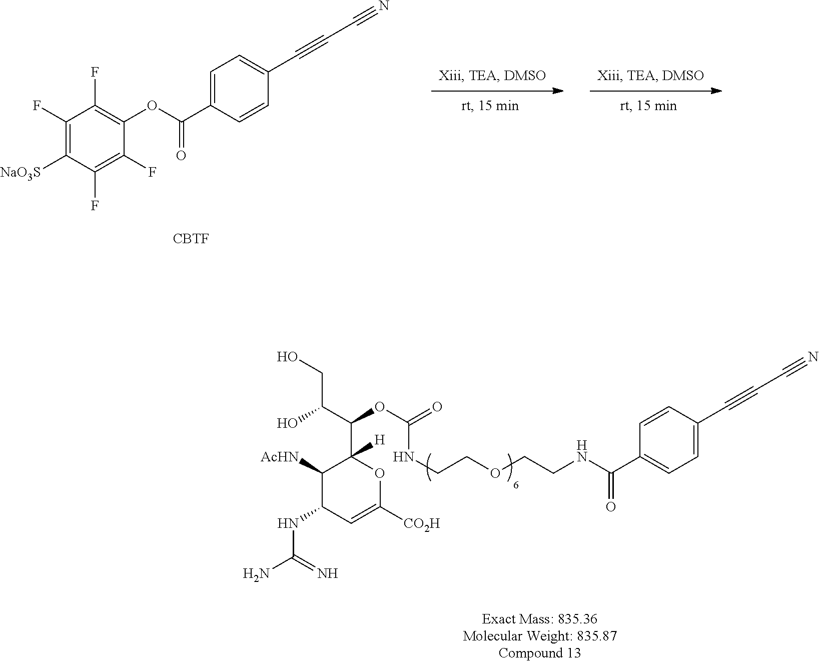

Zanamivir-azide, Compound 4, was prepared from sialic acid according to previously reported literature methods (Chandler et al., Synthesis of the potent influenza neuraminidase inhibitor 4-guanidino Neu5Ac2en. X-ray molecular structure of 5-acetamido-4-amino-2,6-anhydro-3,4,5-trieoxy-D-erythro-L-gluco-nononic acid, J Chemical Society, Perkin Transactions 1:1173-1180 (1995); Shidmoossavee et al., Chemical insight into the emergence of influenza virus strains that are resistant to Relenza, J American Chemistry Society 135 (36): 13254-13257 (2013); Ying, L. & Gervay-Hague, One-bead-one-inhibitor-one-substrate screening of neuraminidase activity, ChemBioChem 6 (10): 1857-1865 (2005). FIG. 5 shows an illustrative schematic of such methods.

Synthesis of Compound 5: To a solution of zanamivir-azide (Compound 4) (5 g, 11 mmol) in THF (40 mL), triphenylphosphine (3.67 g, 14 mmol, 1.27 equiv.) was added, and the resulting solution was stirred at rt for 12 hours. Subsequently, water (10 mL) was added, and the solution was stirred at rt for another 24 hours. The reaction solution was then concentrated, and the crude product was purified by flash column chromatography on a Teledyne CombiFlash Rf+ Lumen (silica gel column, 0-100% EtOAc in hexanes) to give Compound 5 as a yellow powder. Product (Compound 5) isolated, 2.89 g; yield, 61.1%.

Synthesis of Compound 6: To a solution of Compound 5 (2.74 g, 6.37 mmol) and N,N′-bis(tertbutoxycarbonyl)-1H-pyrazole-1-carboxamidine (2.57 g, 8.28 mmol, 1.30 equiv.) dissolved in THF (20 mL) was added triethylamine (1.5 mL). The reaction mixture was stirred overnight at rt and then concentrated and purified by flash column chromatography on a Teledyne CombiFlash Rf+ Lumen (silica gel column, 0-100% EtOAc in hexanes) to give Compound 7 as a white solid (4.14 g, 97%).

Synthesis of Compound 8: NaOMe (2.9 mL, 0.5 M, 1.414 mmol) was added to a stirred solution of compound (Compound 6) (4.225 g, 6.281 mmol) in anhydrous methanol (70 mL). The reaction mixture was then stirred for 1 hour. Dowex 50XW8 (H⁺) resin was added to neutralize the reaction mixture, and the mixture was filtered and concentrated to give lead Compound 7, which was used for the next step without further purification.

To the Compound 7 in dry acetone (70 mL) was added 2,2-dimethoxypropane (7.7 mL, 6.54 g, 62.81 mmol, 10 equiv.), followed by p-toluenesulfonic acid (120 mg, 0.628 mmol, 0.1 equiv.). The resulting mixture was stirred overnight at rt. The reaction mixture was then concentrated and purified by flash column chromatography on a Teledyne CombiFlash Rf+ Lumen (silica gel column, 0-100% EtO Ac in hexanes) to give compound Compound 8 as a white solid (2.4 g, 65.2%).

Synthesis of Compound 9: To a solution of Compound 8 (1.46 g, 2.49 mmol) in pyridine (30 mL) were added 4-dimethylaminopyridine (2.13 g, 17.43 mmol) and 4-nitrophenylchloroformate (3.51 g, 17.43 mmol). The reaction mixture was stirred overnight at rt and then concentrated and purified by flash column chromatography on a Teledyne CombiFlash Rf+ Lumen (silica gel column, 0-100% EtOAc in hexanes) to give activated-Zanamivir (Compound 9) as a white solid (1.62 g, 87%).

Synthesis of Compound 10: To a solution of activated-Zanamivir (Compound 9) (0.2 g, 0.27 mmol) in THF (12.0 mL) were added tert-Boc-N-amido-PEG6-amine (0.124 g, 0.29 mmol, 1.1 equiv.) followed by DIPEA (0.23 mL, 1.33 mmol, 5.0 equiv.) at rt under argon with stirring for 6-12 hour. Progress of the reaction was monitored by TLC and LC/MS. After completion of the reaction, the reaction mixture was concentrated under reduced pressure. The crude product was purified by flash column chromatography on a Teledyne CombiFlash Rf+ Lumen (silica gel column, 0-20% MeOH in DCM) to give Compound 10 as a gummy solid (234 mg, 85%).

Synthesis of Compound 12: Compound 10 (0.22 g, 0.21 mmol) was dissolved in THF (1.5 mL) and treated drop-wise with 1 M NaOH (0.6 mL). The reaction mixture was stirred at rt for 1 hour at which time liquid chromatography-mass spectrometry (LC-MS) analysis revealed that deprotection of methyl ester was completed. The reaction mixture was neutralized by adding Dowex® 50WX8 (H⁺) resin, filtered, and concentrated under reduced pressure. The intermediate crude Compound 11 was used directly for the next step without further purification.

TFA (1.5 mL) was added to the intermediate crude Compound 11. The solution was stirred for 1 hour at rt until the reaction was completed as demonstrated by LC-MS. TFA was removed by rotary evaporation under reduced pressure and treated with diethyl ether (3×2 mL) and dried under vacuum to give Compound 12 as a gummy product. The total yield over the 2 steps was 79%.

Synthesis of Compound 13: To a solution of Compound 12 (4 mg, 5.86 μmol) in DMSO (200 μL) were added sodium 4-((4-(cyanoethynyl)benzoyl)oxy)-2,3,5,6-tetrafluorobenzenesulfonate and CBTF (2.60 mg, 6.15 μmol, 1.05 equiv.), followed by triethylamine (8.20 μL, 58.59 μmol, 10.0 equiv.), at rt under argon and stirring for 10-15 minutes. Progress of the reaction was monitored by TLC and LC-MS. After completion of the reaction, Compound 13 was used directly for the next step without any purification (it is better to purify using silica gel column for conjugation with Fc protein).

For the LC-MS evaluations, the conditions were as follows: Column: XBridge BEH C18 Column, 130 Å, 3.5 μm, 3 mm×100 mm; Mobile phase: A: 20 mM ammonium bicarbonate buffer, pH 7; B; Acetonitrile (HPLC Grade); Method run: 5-95% B, 7 minutes, 0.75 mL/minutes.

WT-Fc Expression, purification, and QC analysis: The IgG1 C_{H}2-C_{H}3 WT was synthesized and sub-cloned into an expression vector using known methods. The confirmed plasmid DNA was prepared and used to transfect transiently CHO—S cells. Five to six days after transfection, the cell suspension was centrifuged at 8,000 rpm for 30 minutes to recover the supernatant fraction. The IgG1 Fc WT (WT-Fc) was purified by protein A affinity chromatography.

Briefly, the culture supernatant was passed through 0.22 μm filters before loading onto polypropylene columns packed with Protein A high-capacity agarose resin. The resulting flow-through was collected and passed twice more through the column before any unbound protein was washed away with >10 CV (Column Volume) of 1× PBS. The WT-Fc was eluted with 3 ml of 100 mM citrate buffer (pH 3.0) and immediately neutralized with 1 ml of 1 M Tris (pH 8.0). Samples were buffer-exchanged into 1× PBS using Amicon Ultra-4 (Millipore) spin columns with a 10 kDa cutoff, and the purity of purified samples was assessed by 4-20% gradient SDS-PAGE gel. The final Fc protein was approximately 26 kDa as monomeric form. The yield is around 30 to 40 mg per 100 ml culture.

Final cysteine-based conjugation for Zan-PEG₆-Fc-WT: To the purified Fc protein in PBS solution, pH 7.2, was added Compound 13 (10-12 equiv.) while stirring slowly at 4° C. for 10 minutes. After completion of the addition, the reaction mixture was stirred for 12-72 hours and progress of the reaction was monitored by SDS-PAGE and Matrix Assisted Laser Desorption/Ionization (MALDI) mass analysis.

After completion of reaction, the conjugated crude product was purified using molecular weight cut off (MWCO; 10 kDa, Vivaspin 500, Catalog #GE28-9322-25) filters and centrifugation at 4° C., 15,000/RPM, 10 minute to remove all unreacted linker as well as low molecular weight impurities. The process was repeated 3-5 times (at 5 mg/mL concentration, yield: 60-70%).

The purity and molecular weight of zan-PEG₆-Fc-WT conjugate was confirmed by SDS-PAGE and MALDI analysis, respectively. The final product molecular weight was ˜29 kDa, whereas the molecular weight for the Fc-protein alone was 26 kDa.

The above protocol was followed for two other final cysteine-based conjugates, zan-PEG₆-Fc-DLE (SEQ ID NO: 6 attached to zanamivir via a PEG₆ linker; also referred to herein as “Zan-Mono-Fc-DLE”) and zan-PEG₆-Fc-GASDALIE (SEQ ID NO: 5 attached to zanamivir through via a PEG₆ linker; also referred to herein as “Zan-Mono-Fc-GASDALIE”), and the products were confirmed by SDS-PAGE and MALDI analysis. Further, tyrosine-based conjugation was carried out using the same procedures with another activating zan-PEG₆-Linker.

### Example 4

### Cell-Based ADCC Assay

Zan-Fc monomers surrogate ADCC evaluation: The Zan-Mono-Fc-WT (SEQ ID NO: 4 attached to zanamivir via a PEG₆ linker; also referred to herein as “Zan-PEG₆-Fc-WT”), Zan-Mono-Fc-DLE (SEQ ID NO: 6 attached to zanamivir via a PEG₆ linker), and Zan-Mono-Fc-GASDALIE (SEQ ID NO: 5 attached to zanamivir via a PEG₆ linker) functions were evaluated using the Promega® ADCC reporter assay (Cat #G7010; Promega Corporation, Madison, Wisconsin) according to the manufacturer's instructions using the following detailed setup: Plate preparation:
1. Preparation of poly-D-Lysine (Sigma Aldrich #501779548; Sigma Aldrich, Burlington, Massachusetts): 100 ml of sterile tissue culture grade H₂O were added to 5 mg poly-D-lysine to give a final concentration of poly-D-lysine of 50 μg/ml. The solution was mixed by pipetting several times and filter-sterilized, and subsequently was stored at 2-8° C. or −20° C.
2. Preparation of coated plates (CulturPlate-96, White Opaque 96-well Microplate, Sterile and Tissue Culture Treated (PerkinElmer 6005680; PerkinElmer Inc., Waltham, Massachusetts): 150 μL of poly-D-lysine solution was added to cover the surface of the culture plate. The plate was incubated at rt for 1 hour, followed by aspiration of the solution. The plate surface was rinsed thoroughly with sterile tissue culture grade H₂O, followed by drying and storage at 4-8° C. or room temperature.

One day before the assay was performed, WT and NA-expressing HEK293 healthy target cell plates (confluency 80 to 90%) were trypsinized and cells were counted and suspended at an appropriate cell density (0.1×10⁶ cells/ml). 100 μl of cells (10,000 cells) were transferred to the wells of white 96-well assay plates using a multichannel pipette. Lids were placed on the plates and the plates were incubated overnight (20 hours) in a CO₂ incubator at 37° C. Target cells were seeded in a 96-well plate (CulturPlate-96, White Opaque 96-well Microplate, Sterile and Tissue Culture Treated (Perkin Elmer 6005680; PerkinElmer Inc., Waltham, Massachusetts).

On the morning of the assay, a multichannel pipette was used to remove 95 μl of culture medium from each of the wells, and the wells were gently washed twice with 100 μl of PBS. The pipette tips were touched to the walls of the wells and buffer was added gently to minimize cell disruption. 50 μl of ADCC Assay Buffer (prewarmed to 37° C.) was added to the wells. The pipette tips were touched to the walls of the wells, and buffer was added gently to minimize cell disruption.

4× concentrated, 5-fold serial dilutions in ADCC assay buffer were aseptically prepared, and 25 μl were gently added to the wells. The pipette tips were touched to the wall of the wells, and buffer was added gently to minimize cell disruption. The plates were then incubated at rt for 30 minutes.

A sterile 15 ml conical tube was labeled “ADCC Bioassay Effector Cells.” 3.6 ml of ADCC Assay Buffer (prewarmed to 37° C.) were added to the tube. One vial of ADCC Bioassay Effector Cells was removed from −140° C. freezer storage or vapor phase of liquid nitrogen to dry ice for transport to the bench on the day of use. The vial was thawed in a 37° C. water bath until the cells were just thawed (about 2-3 minutes). While thawing, the cells were gently agitated and visually inspected. The vial was not inverted.

The cell suspension was gently mixed by pipetting 1 or 2 times. 630 μl of cells were transferred to the 15 ml “ADCC Bioassay Effector Cells” tube containing 3.6 ml of ADCC Assay Buffer, which was mixed well by gently inverting the tube 2 times. The cell suspension was then transferred to a sterile reagent reservoir. Immediately, using a multichannel pipette, 25 μl of cells were added to the right wells of the 96-well assay plates already containing target cells.

The plates were covered with lids, and the plates were incubated for 6 hours at 37° C. in a humidified CO₂ incubator. Plates were not stacked in the incubator.

Assay plates were removed from the 37° C. incubator and equilibrated to ambient temperature (22-25° C.) on the bench for 15 minutes. Using a manual multichannel pipette, 100 μl of Bio-Glo™ Luciferase Assay Reagent were added to all the inner 60 wells of the assay plates, avoiding the creation of bubbles. The plates were then incubated at ambient temperature for 5-30 minutes.

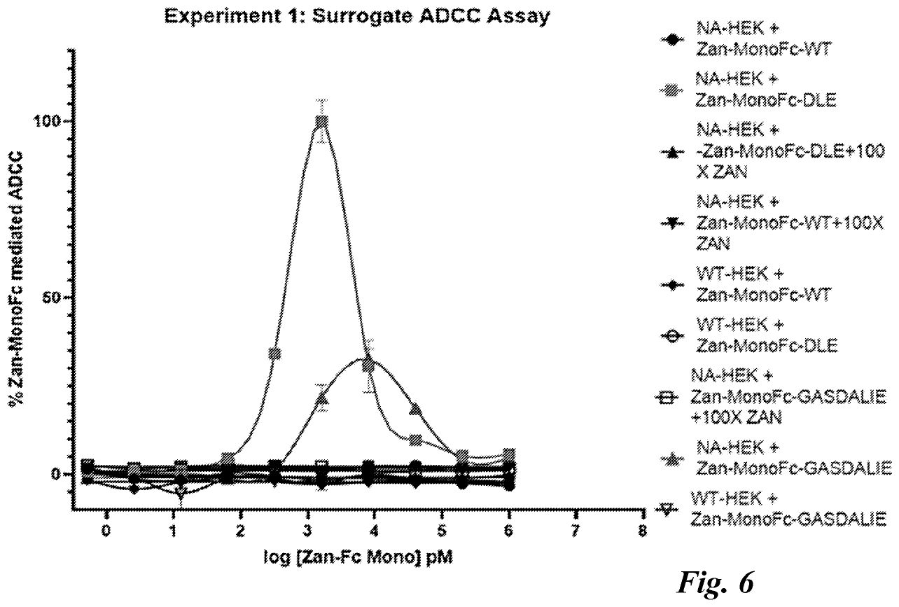

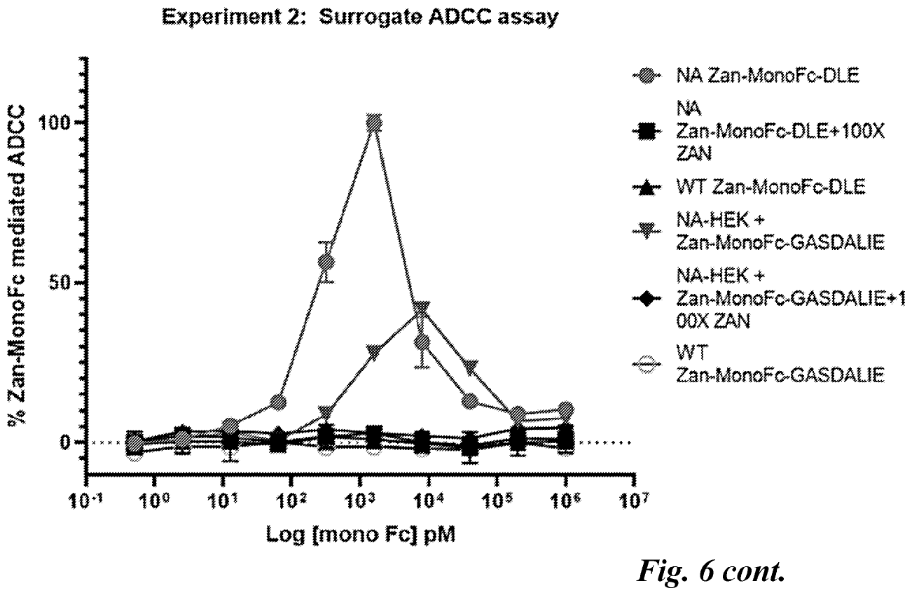

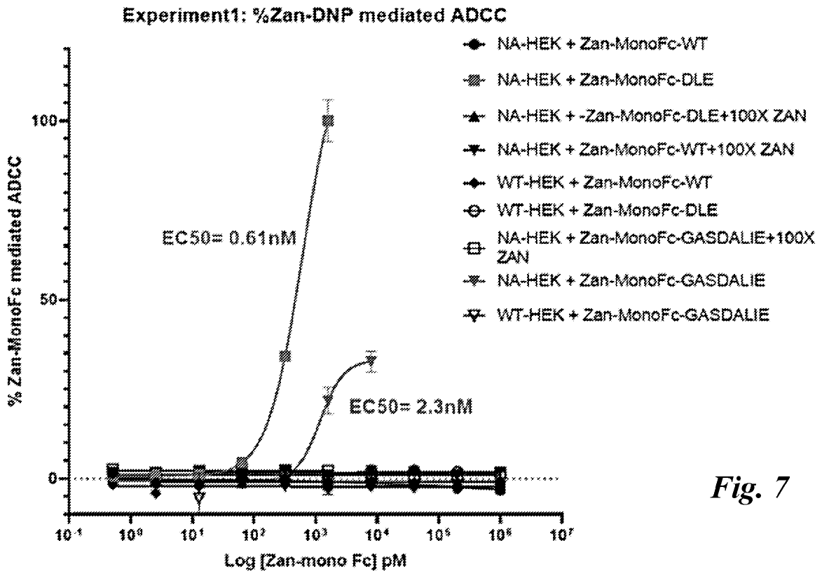

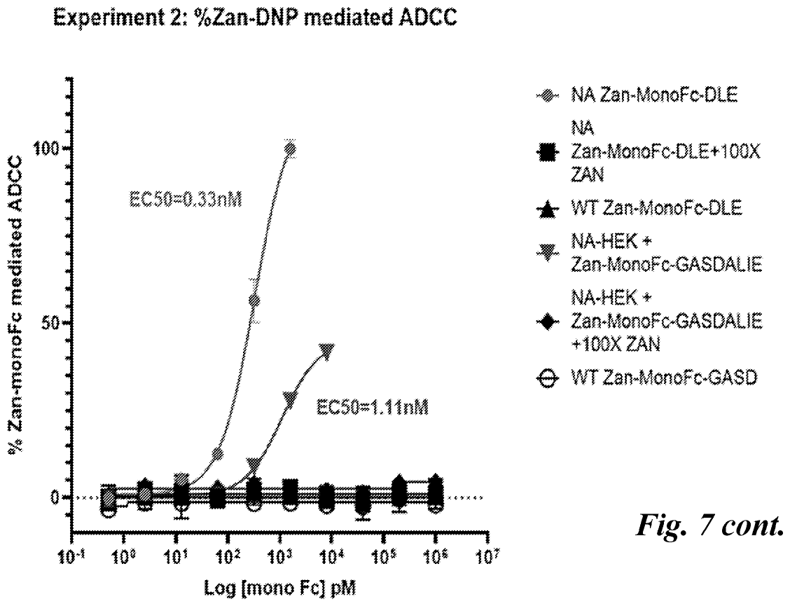

Luminescence was measured using a Bio-Tek plate reader with glow-type luminescence read capabilities. The results are shown in FIGS. 6 and 7. FIG. 6 shows surrogate ADCC assay data, whereas FIG. 7 shows ADCC assay data. The figures show the percentage of luciferase activity data normalized to the highest response obtained in the assay from Zan-MonFc variants in the presence and absence of 100× Zanamivir. In particular, the highest response in Experiment 1 of FIG. 6 is the curve associated with “NA-HEK+Zan-MonoFc-DLE” with the second highest response being the curve associated with “NA-HEK+Zan-MonoFC-GASDALIE”. The highest response in Experiment 2 (FIG. 6 cont.) is the curve associated with “NA-Zan-MonoFc-DLE” with the second highest response being the curve associated with “Na-HEK+Zan-MonoFc-GASDALIE.

The data in FIG. 6 represent two independent ADCC assays performed to ensure reproducibility of the results. A characteristic bell-shaped curve was observed in both experiments and it clearly demonstrates that both variants Zan-Mono-Fc-DLE and Zan-Mono-Fc-GASDALE can activate the ADCC effector cells with an average EC₅₀ value of 0.47 nM and 1.71 nM, respectively, in comparison to the WT, which was calculated from the data by nonlinear regression analysis performed using PRISM 9 software. Likewise, the data in FIG. 7 show the highest responses with “NA-HEK+Zan-MonoFc-DLE” (EC₅₀ of 0.61 nm) and “NA-HEK+Zan-MonoFc-GASDALIE” (EC₅₀ of 2.3 nm). The addition of 100× Zan suppressed this activation, demonstrating the specificity of the conjugates in triggering the ADCC response. The data overall suggests the ability of these conjugate variants to bind specifically to neuraminidase on the surfaces of target cells through the Zan portion, while engaging the activation of the effector cells expressing CD16a (V) via the Fc portion of the molecules. Note that the terms “Zan-MonoFc-DLE”, “zan-FC (DLE)” and “Zan-Fc-DLE” are synonymous, as are the terms “Zan-MonoFc-GASDALIE” and “zan-Fc (GASDALIE)” throughout the disclosure.

### Example 5

### Testing the Zanamivir-Fc Conjugates In Vivo

Female Balb/c mice (6-8 weeks old) were infected with 10 LD₅₀ of influenza A/H1N1/PR8/1934 virus. 48 hours after infection, mice were treated with 50 μg of the respective zanamivir-Fc conjugates (WT/DLE/GASDALIE) in three experimental groups (n=5/group). The mice in the control group received saline only. Body weights of the mice were measured daily as the experimental readout.

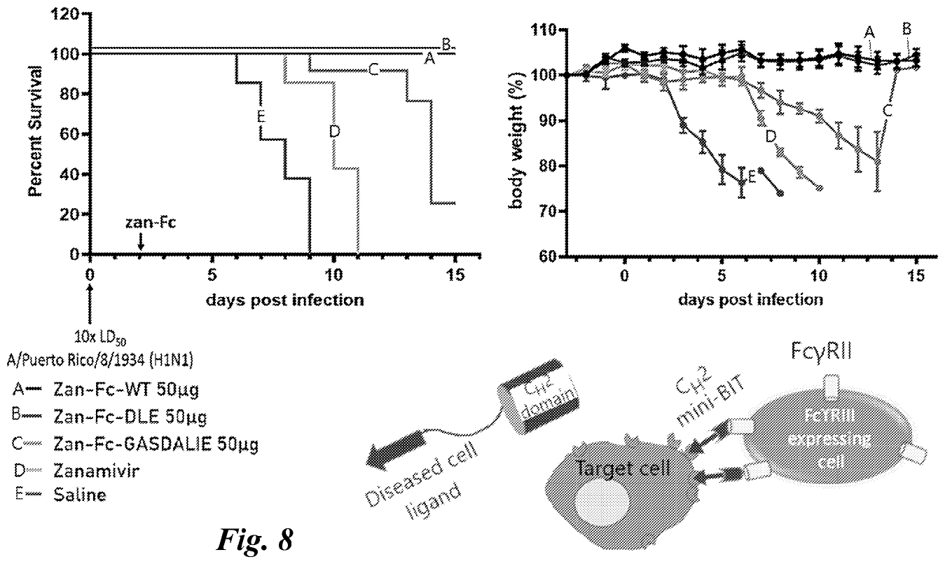

FIG. 8 shows the treatment of influenza A/H1N1/PR8-infected mice with zan-Fc conjugate. The groups treated with zan-Fc (WT) and zan-Fc (DLE) had a 100% survival rate, the group treated with zan-Fc (GASDALIE) had a 20% survival, and no mice survived in the control group.

### Example 6

Head-to-Head Comparison of the Zan-Fc Conjugates with Other Payloads

To compare the efficacy of zan-Fc with other zan-payload conjugates and the commercially available flu drug, Xofluza, a study was conducting using the protocol set forth in Table 4 and as described below.

TABLE 4
Experimental design for the head-to-head payload comparison study

| | | | | | Route of |
|---|---|---|---|---|---|
| Group | Mouse # | Test Article | Dose Concentration | | Administration |
| 1 | 17 | Xofluza | Xofluza (12.3 mg/kg single | | Oral gavage (OG) |
| | (5 × 3 + 2) | | dose) | | |
| 2 | 17 | Zan-PEG₆-DNP | 1.5 μmol/kg + 3 mg/kg | | Intravascular (IV) |
| | | | anti-DNPAb (Polyclonal | | (zan-DNP) + |
| | | | IgG/Rabbit) | | IV (anti-DNPAb) |
| 3 | 17 | Zan-PEG₆- | 1.5 μmol/kg (5 doses) | | Intranasal (IN) |
| | | DBCO- | | | |
| | | PEG₆-folate | | | |
| 4 | 14 | Zan-PEG₆-folate | 1.5 μmol/kg (5 doses) | | OG |
| | (4 × 3 + 2) | | | | |
| 5 | 17 | Zan-Fc-WT | 50 | μg | IV |
| 6 | 17 | Zan-Fc-DLE | 50 | μg | IV |
| 7 | 6 (4 + 2) | Saline | 100 | μL | IV |

Briefly, 6-8 weeks old female Balb/c mice were infected with 10 LD₅₀ of influenza A/H1N1/PR8/1934 virus. Each test article identified in Table 3 was administered at 48, 72 and 96 hours post-infection (hpi) in three respective experimental groups based on when the therapy was administered. The dosage and route of administration for each test article are indicated in Table 4.

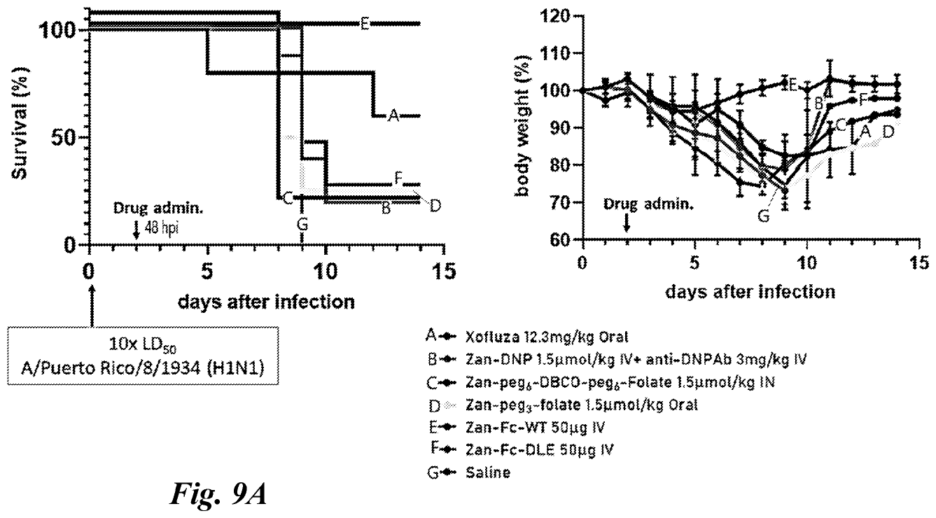

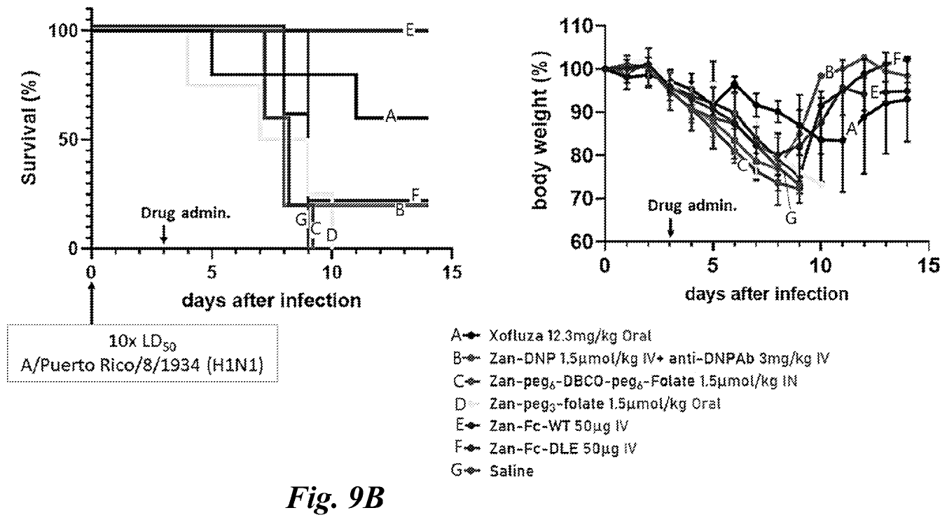

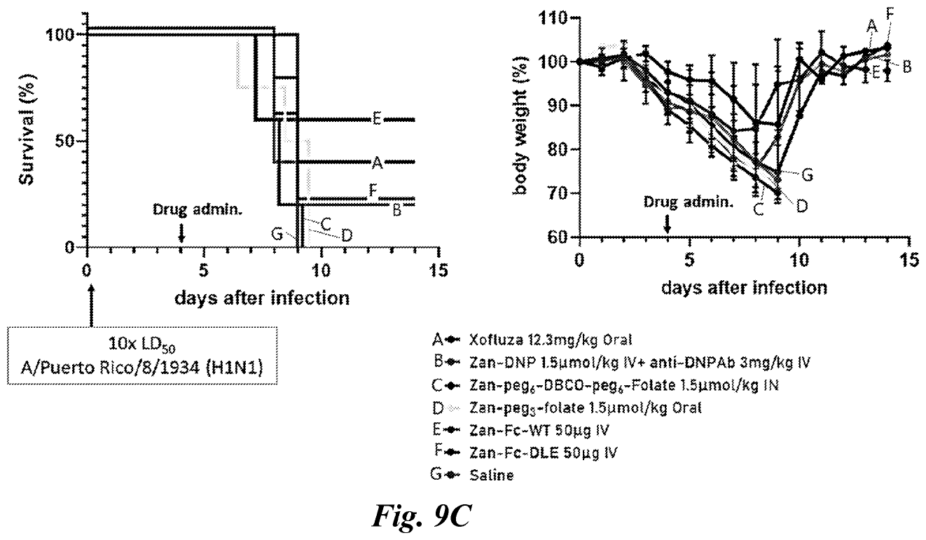

The results are shown in FIGS. 9A-9C. FIG. 9A shows treatment of influenza A/H1N1/PR8-infected mice with different conjugates beginning 48 hpi, whereas FIG. 9B shows treatment beginning 72 hpi, and FIG. 9C shows treatment beginning 96 hpi. Single doses of conjugates were administered for each group, except five doses of zan-folates were administered.

1. A monomeric Fc (mFc) fragment of human immunoglobulin G1 (IgG1) which:
(i) has a molecular weight of less than 40 kDa,
(ii) comprises (a) a C_{H}2 domain, (b) a C_{H}2 domain to the C terminus of which a neonatal Fc receptor (FcRn) binding motif from a C_{H}3 domain is attached, (c) a C_{H}3 domain, or (d) both of a C_{H}2 domain and a C_{H}3 domain,
(iii) binds to the FcRn and at least one Fcγ receptor, and
(iv) comprises SEQ ID NO: 1 at an N-terminus of the fragment, optionally substituted with one or two conservative amino acid substitutions.

2-29. (canceled)

30. A conjugate of formula I:
mFc-L-smL (Formula I)
or a pharmaceutically acceptable salt thereof, wherein:
mFc is a monomeric Fc fragment of human immunoglobulin G1 (IgG1) which:
(i) has a molecular weight of less than 40 kDa,
(ii) comprises (a) a C_{H}2 domain, (b) a C_{H}2 domain to the C terminus of which a neonatal Fc receptor (FcRn) binding motif from a C_{H}3 domain is attached, (c) a C_{H}3 domain, or (d) both of a C_{H}2 domain and a C_{H}3 domain,
(iii) binds to the FcRn and at least one Fcγ receptor, and
(iv) comprises SEQ ID NO: 1 at an N-terminus of the fragment, optionally substituted with one or two conservative amino acid substitutions;
smL is a radical of a small molecule ligand that binds to neuraminidase (NA) or hemagglutinin (HA) on influenza virus or an influenza virus-infected cell; and
L is a linker that is covalently bound to smL and mFc.

31. The conjugate of claim 30, where smL is a radical of a NA inhibitor or an HA inhibitor.

32. The conjugate of claim 30, wherein smL is a radical of zanamivir.

33. The conjugate of claim 30, wherein smL is a radical of oseltamivir, peramivir, or laninamivir.

34. The conjugate of claim 30, wherein mFc comprises at least one cysteine, lysine, and/or tyrosine via which L is covalently bound to mFc.

35. The conjugate of claim 30, wherein L is covalently bound to mFc via an unnatural amino acid.

36. The conjugate of claim 30, wherein L further comprises a spacer.

37. The conjugate of claim 30, wherein L is non-cleavable.

38. The conjugate of claim 30, wherein L increases the water-solubility of the conjugate.

39. The conjugate of claim 30, wherein L comprises one or more of an amino acid, a polyethylene glycol (PEG) monomer, a PEG oligomer, a PEG polymer, or a combination of two or more of the foregoing.

40. The conjugate of claim 30, wherein L comprises an oligomer of peptidoglycans, glycans, anions, or a combination of two or more of the foregoing.

41-44. (canceled)

45. A method of treating a subject having viral infection or a cell infected with a virus comprising administering to the subject an effective amount of a conjugate of claim 30 or a composition comprising the conjugate and a pharmaceutically acceptable carrier.

46. The method of claim 45, wherein the immune effector is the mFc fragment of claim 1.

47. The method of claim 45, wherein the conjugate or composition is administered intravascularly, orally, or intranasally.

48. The method of claim 45, wherein the conjugate or composition is administered to the subject in a single dose.

49. The method of claim 45, wherein the conjugate or composition is administered to the subject in more than one dose.

50. The method of claim 45, wherein the virus is an Influenza virus.

51. The method of claim 45, wherein the virus is an Influenza A virus.


SEQUENCE LISTING (19 sequences)

SEQ ID NO: 1 - protein, 15 aa, Homo sapiens
EPKSGDKTHTSPPGP

SEQ ID NO: 2 - protein, 5 aa, Homo sapiens
HNHYT

SEQ ID NO: 3 - protein, 14 aa, Homo sapiens
FGNCAYLRPHNHYT

SEQ ID NO: 4 - protein, 232 aa, Homo sapiens
EPKSGDKTHTSPPGPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 232 aa, synthetic construct
EPKSGDKTHTSPPGPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, synthetic construct
EPKSGDKTHTSPPGPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 232 aa, synthetic construct
EPKSGDKTHTSPPGPAPELLGGPSVFCFPPKPKDTLMISRTPEVTCVVVDVSHEDPEVKFNWYVDGVEVHNAKTKPREEQYNSTYRVVSVLTVLHQDWLNGKEPKCKVSNKALPAPIECTISKAKGQCREPQVYTSPPSRDELTKNQVSLRCHVKGFYPSDIAVEWESNGQPENNYKTTKPVLDSDGSFFLYSKLTVDKSRWQQGNVFSCSVMHECLHNHYTQKSLSLSPGK

SEQ ID NO: 8 - protein, 232 aa, synthetic construct
EPKSGDKTHTSPPGPAPELLGGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPLPEECTISKAKGQCREPQVYTSPPSRDELTKNQVSLRCHVKGFYPSDIAVEWESNGQPENNYKTTKPVLDSDGSFFLYSKLTVDKSRWQQGNVFSCSVMHECLHNHYTQKSLSLSPGK

SEQ ID NO: 9 - protein, 233 aa, synthetic construct
EPKSGDKTHTSPPGPAPELLAGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPLPEECTISKAKGQCREPQVYTSPPSRDELTKNQVSLRCHVKGFYPSWDIAVEWESNGQPENNYKTTKPVLDSDGSFFLYSKLTVDKSRWQQGNVFSCSVMHECLHNHYTQKSLSLSPGK

SEQ ID NO: 10 - protein, 232 aa, synthetic construct
EPKSGDKTHTSPPGPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSLSPGK

SEQ ID NO: 11 - protein, 233 aa, synthetic construct
EPKSGDKTHTSPPGPAPELLGGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPLPEECTISKAKGQPREPQVYTSPPSRDELTKNQVSLRCHVKGFYPSWDIAVEWESNGQPENNYKTTKPVLDSDGSFFLYSKLTVDKSRWQQGNVFSCSVMHEALHNHYTQKSLSLSPGK

SEQ ID NO: 12 - protein, 232 aa, synthetic construct
EPKSGDKTHTSPPGPAPELLAGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPLPEECTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSLSPGK

SEQ ID NO: 13 - protein, 139 aa, synthetic construct
EPKSGDKTHTSPPGPAPELLGGPSVFCFPPKPKDTLMISRTPEVTCVVVDVSHEDPEVKFNWYVDGVEVHNAKTKPREEQYNSTYRVVSVLTVLHQDWLNGKEPKCKVSNKALPAPIECTISKAKFGNCAYLRPHNHYT

SEQ ID NO: 14 - protein, 139 aa, synthetic construct
EPKSGDKTHTSPPGPAPELLGGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPLPEECTISKAKFGNCAYLRPHNHYT

SEQ ID NO: 15 - protein, 139 aa, synthetic construct
EPKSGDKTHTSPPGPAPELLAGPDVFCFPPKPKDTLMISRTPEVTCVVVDVSHEDPEVKFNWYVDGVEVHNAKTKPREEQYNSTYRVVSVLTVLHQDWLNGKEYKCKVSNKALPLPEECTISKAKFGNCAYLRPHNHYT

SEQ ID NO: 16 - protein, 139 aa, Homo sapiens
EPKSGDKTHTSPPGPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKFGNCAYLRPHNHYT

SEQ ID NO: 17 - DNA, 696 bp, Homo sapiens
  source 1..696: mol_type = genomic DNA
gagcccaagagcggcgacaagacccacaccagcccccccggccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 18 - DNA, 696 bp, synthetic construct
  source 1..696: mol_type = other DNA
gagcccaagagcggcgacaagacccacaccagcccccccggccccgcccccgagctgctggccggccccgacgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccctgcccgaggagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 19 - DNA, 696 bp, synthetic construct
  source 1..696: mol_type = other DNA
gagcccaagagcggcgacaagacccacaccagcccccccggccccgcccccgagctgctgggcggccccgacgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccctgcccgaggagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag